(19) United States
(12) Patent Application Publication
(10) Pub. No.: US 2008/0312140 A1
(43) Pub. Date: Dec. 18, 2008

(54) Compositions and Methods for Preventing or Treating Hiv Infection

(76) Inventors: Chaim Gilon, Jerusalem (IL); Shira Cohen, Modi'in (IL); Christian Devaux, Montpellier (FR); Laurence Briant-Longuet, Gallaugues (FR); Martine Bardy, Montpellier (FR)

Correspondence Address: WINSTON & STRAWN LLP;PATENT DEPARTMENT, 1700 K STREET, N.W., WASHINGTON, DC, 20006, US

(21) Appl. No.: 11/916,519

(22) PCT Filed: Jun. 22, 2006
(86) PCT No.: PCT/IL2006/000741

§ 371 Date: Aug. 20, 2008

Related U.S. Application Data
(60) Provisional application No. 60/693,057, filed on Jun. 23, 2005.

(51) Int. Cl. A61K 38/02; C07K 2/00; A61P 31/18

(52) U.S. Cl. 514/9; 530/317

(57) ABSTRACT

The present invention is directed to compositions and methods of preventing for treating a retroviral infection, more particularly an HIV (human immunodeficiency virus) infection, and even more particularly an HIV-1 infection. The compositions and methods involve molecules that mimic the active site of the human CD4 protein.

### FIELD OF THE INVENTION

The present invention is directed to compositions and methods for preventing or treating a retroviral infection, more particularly a human immunodeficiency virus (HIV) infection, and even more particularly an HIV-1 infection. The compositions and methods involve backbone cyclized molecules that mimic the gp120-binding site of the human CD4 protein.

### BACKGROUND OF THE INVENTION

The HIV retrovirus is responsible for AIDS (acquired immunodeficiency syndrome), an incurable disease in which the body's immune system breaks down leaving it vulnerable to opportunistic infections, such as pneumonia, and certain cancers, such as Karposi's sarcoma. AIDS is a major global health problem. Recent studies estimate over 34 million people with HIV. AIDS has killed nearly 25 million people, has replaced malaria and tuberculosis as the world's deadliest infectious disease, and is the fourth leading cause of death in the world.

AIDS remains a major disease that is elusive of a cure after almost two decades of intense search for an effective treatment. Currently available HIV drugs include reverse transcriptase (RT) and protease inhibitors (PR). Although drug combination regimens has results in significant decline of AIDS related death in the developed world, 78% of HIV patients with measurable viral loads carry virus that is resistant to one or more drugs. Furthermore, many of the newly diagnosed HIV patients are infected with resistant viruses. Compounds with novel anti-HIV targets are therefore required. Agents that interfere with HIV entry into the cell represent one class of inhibitors suggested for treating HIV infections (D'Souzaet al., 2000, JAMA 284, 215-222).

The major problem in developing an efficient drug against AIDS is the virus tendency to mutate. Since HIV is an organism with relatively primitive control mechanisms, this virus, like many other retroviruses, tends to have a high mutation rate. This high mutation rate causes frequent generation of various viral types, so when exposed to the drugs in use, shortly a resistant type is formed. Thus, one of the challenges facing researchers today is developing an irresistible anti HIV drug. A drug of this sort should target a conserved viral site. However, any mutation in the viral site could lead the drug to becoming non-functional.

### CD4 and CD4 Mimetics

HIV envelope consists of an exterior glycoprotein gp120 and a transmembrane domain gp41. The HIV entry process involves the initial contact between the gp120 and the host cell CD4 receptor (Doms, R. W. and Moore, J. P., 2000, J. Cell. Biol. 151, F9-F14.). Subsequent conformational changes facilitate the binding of gp120 to the coreceptor CCR5 or CXCR4 and the insertion of the fusion peptide into the host membrane, finally resulting in fusion of the virus and cell membranes.

CD4 is a mostly extra-cellular co-receptor embedded in the T cell membrane by a trans-membranal domain, followed by a short intra-cellular domain. This protein is very important in proper function of the immune system, mainly in the binding of CD4+ T cells to antigen presenting cells.

Agents targeting the HIV entry process are categorized into three groups based on the mode of action: (I). GP120/CD4 binding inhibitors; (II). Co-receptor inhibitors and (III). GP41 fusion peptide inhibitors. The truncated form of CD4 (sCD4) competes with the cell associated CD4 receptor for gp120 binding, therefore the protein exhibited potent antiviral activity against HIV-1. Yet, initial efforts to develop soluble CD4 as an anti-HIV agent failed due to its short serum half-life and its lack of activity against clinical HIV-1 isolates (Daar et al., 1990, Proc. Natl. Acad. Sci. USA 87, 6574-6578).

The recombinant CD4-Ig fusion proteins PRO542 produced by Progenic Pharmaceuticals demonstrated improved half-life in blood and achieved inhibitory activity over a broad range of HIV subtypes (Jacobson et al., 2000, J. Infect. Dis. 182, 326-329) and this compound has entered phase II trial in an IV formulation. Other CD4 peptide mimics have been shown to have affinities to gp120 too weak to produce significant anti-HIV activity.

The crystal structure of a ternary complex composed of gp120 with the V1V2V3 loop-deleted the DlD2 domain CD4 and the Fab fragment of 17b (a CD41 monoclonal antibody) has been reported (Furuta et al., 1998, Nat. Struct. Biol. 5, 276-279).

The most important residue in the CD4-gp120 binding site is CD4's Phe43. This residue is situated on a type II′β-turn and its phenyl ring enters a hydrophobic pocket in gp120. This residue is responsible for 23% of the binding interactions between the two proteins, either by hydrophobic interactions of its phenyl ring or by both hydrophobic and hydrophilic interactions of its backbone atoms. It interacts with many gp120 residues: Glu370, Ile371, Asn425, Met426, Trp427, Gly473 and Asp368. Only the interaction with Ile371 is a classical hydrophobic one. There is also an aromatic stacking interaction of its phenyl ring with the carboxylate group of Glu370. Other interactions involve backbone atoms only. The second important residue is Arg59 of CD4. This residue forms a hydrogen bond with Asp368 of gp120. Residues Lys46, Lys35 and Lys29 are less important. Residues Asp368, Glu370 and Trp427, as well as the residues forming the hydrophobic pocket of gp120, were found to be conserved amongst various HIV strains. This shows their high importance in activity. A few point mutations were found to increase the binding affinity of the two proteins. Replacing Arg59 with a Lys residue tripled binding affinity, while replacing Gln40 or Asp63 by Ala doubles it.

PCT patent application WO 99/24065 discloses some theoretical inhibitors that could interfere with gp120/CD4 interaction through binding with the amino acid residues located in the D1D2-CD4 binding region of gp120. The possible inhibitors claimed are purely theoretical at this time. The inventors of WO 99/24065 have so far failed to produce any of the inhibitors disclosed in the PCT publication possessing the specified chemical characteristics and anti-HIV activity.

US Patent Application published as US 20040162298 describes a method of inhibiting HIV infection in a mammal by administering a small molecule compound having a molecular weight of less than about 1,000 dalton, wherein the compound interacts with HIV-gp120 and cause conformational change in the gp120 thereby preventing interaction between said gp120 and leukocyte CD4. The invention is exemplified by use of three small molecule compounds BMS-216, BMS-853 and BMS-806 disclosed in U.S. Pat. Nos. 6,469,006 and 6,476,034. The patent disclose that the compounds can be orally administered.

### Improved Peptide Analogs

As a result of major advances in organic chemistry and in molecular biology, many bioactive peptides can now be prepared in quantities sufficient for pharmacological and clinical use. Thus in the last few years new methods have been established for the treatment and diagnosis of illnesses in which peptides have been implicated.

However, the use of peptides as therapeutic and diagnostic agents is limited by the following factors: a) low tissue penetration; b) low metabolic stability towards proteolysis in the gastrointestinal tract and in serum; c) poor absorption after oral ingestion, in particular due to their relatively high molecular mass or the lack of specific transport systems or both; d) rapid excretion through the liver and kidneys; and e) undesired side effects in non-target organ systems, since peptide receptors can be widely distributed in an organism.

It would be desirable to achieve peptide analogs with greater specificity thereby achieving enhanced clinical selectivity. It would be most beneficial to produce conformationally constrained peptide analogs overcoming the drawbacks of the native peptide molecules, thereby providing improved therapeutic properties.

Proteinomimetics are small molecules that mimic the structure and/or the activity of a large parent protein. The availability of such small molecules can be useful for the detailed study of the biological function, molecular structure and folding of proteins. Moreover, proteinomimetics are excellent candidates for becoming a novel type of drugs, since they overcome some of the limitations that currently hamper the therapeutic use of proteins and polypeptides such as antigenicity, metabolic instability and poor bioavailability. While many structural proteinomimetics have already been described, most of them were deprived of the biological function which characterized the parent protein. Also attempts to obtain small peptides which mimic catalytic sites of enzymes and preserve their enzymatic activity have so far failed (Corey and Corey 1996, Proc. Natl. Acad. Sci. USA 93, 11428-11434). Very few examples of structural proteinomimetics which retain the biological activity and resemble the structure of the corresponding proteins have so far been disclosed, such as the zinc-finger (Struthers, et al., 1996 Science 271, 342-345) and the metal-binding proteinomimetics (Robertson, et al., 1994 Nature 368, 425-432; Pessi, et al., 1993, Nature 362, 367-369).

A novel conceptual approach to the conformational constraint of peptides was introduced by Gilon, et al., (Biopolymers, 1991, 31, 745) who proposed backbone cyclization of peptides. Backbone cyclization is a general method by which conformational constraint is imposed on peptides. In backbone cyclization, atoms in the peptide backbone (N and/or C) are interconnected covalently to form a ring.

The theoretical advantages of this strategy include the ability to effect cyclization via the carbons or nitrogens of the peptide backbone without interfering with side chains that may be crucial for interaction with the specific receptor of a given peptide. Further disclosures by Gilon and coworkers (WO 95/33765, WO 97/09344, U.S. Pat. No. 5,723,575, U.S. Pat. No. 5,811,392, U.S. Pat. No. 5,883,293, U.S. Pat. No. 6,265,375 and U.S. Pat. No. 6,407,059), provided methods for producing building units required in the synthesis of backbone cyclized peptide analogs. The successful use of these methods to produce backbone cyclized peptide analogs of bradykinin analogs (U.S. Pat. No. 5,874,529), and backbone cyclized peptide analogs having somatostatin activity (WO 98/04583, WO 99/65508, U.S. Pat. No. 5,770,687, U.S. Pat. No. 6,051,554 and U.S. Pat. No. 6,355,613) was also disclosed.

There remains a need for small molecules which mimic the binding site of the CD4 to the gp120 glycoprotein on the virus's envelope. Desirable molecule should have increased inhibitory activity, in vivo stability and membrane permeability, thereby providing pharmaceutical compounds for the treatment of viral infections, particularly HIV infection. The present invention addresses this need by providing small backbone cyclic peptides which mimic structure and the function of active regions in the CD4 protein.

### SUMMARY OF THE INVENTION

The present invention provides novel compounds, compositions comprising these compounds and methods of using same for preventing or treating a viral infection, particularly an HIV infection. The compounds are backbone-cyclized molecules that mimic the structure and the function of the active region of the human CD4 protein thereby capable of binding to the viral gp120 glycoprotein and inhibiting the virus binding to the cells.

According to the principles of the present invention it is now disclosed that using the backbone cyclic proteinomimetic approach it is possible to design libraries of backbone cyclic peptides that mimic the region of the CD4 protein which bind to the gp120 protein. These libraries can be used to identify molecules which can then be further optimized and refined to have improved inhibitory activity, cells permeability and metabolic stability.

According to one aspect of the present invention, small cyclic proteinomimetics which mimic the binding cite of CD4 to gp120 are provided. According to a specific embodiment the peptidomimetics are backbone cyclized peptide analogs comprising a peptide sequence of three to twelve amino acids that incorporates at least one building unit, the building unit containing one nitrogen atom of the peptide backbone connected to a bridging group comprising a disulfide, amide, thioether, thioester, imine, ether, or alkene bridge, wherein at least said one building unit is connected via the bridging group to a moiety selected from the group consisting of a second building unit, a side chain of an amino acid residue of the peptide sequence, and a N-terminal amino acid residue, to form a cyclic structure.

Preferably, the peptide sequence incorporates three to six amino acids. More specifically, the peptide sequence comprises at least one aromatic residue and at least one positively charged residue. According to specific embodiments the aromatic residue is Phe or D-Phe and the positively charged residue is Arg or D-Arg.

According to certain embodiments, the bridging group in the backbone cyclic peptide analog is a chemical linker having the general Formula I:

—(CH)ₙ—(CH)Y-M-A-B- Formula I

wherein n is an integer for 1 to 8; M is selected from the group consisting of a disulfide, amide, thioether, thioester, imine, ether, or alkene bridge; Y is hydrogen or an amino acid side chain; A is (CH₂)ₘ wherein m is an integer for 1 to 8, or C(R)—NH wherein R is an amino acid side chain; and B is absent or is the residue of a molecule comprising two carboxylic groups.

According to specific embodiment of the present invention the compositions have a structure according to Formula II:

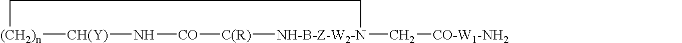

wherein:

Y is hydrogen or is the side chain of Arg, DArg, Lys, DLys, Phe, DPhe, Tic, DTic, Pro, or DPro;

n is 1 to 5;

R is the side chain of Arg, Lys, Phe, DArg, DLys or DPhe;

W₁ is absent or is Phe;

W₂ is absent or is Phe, DPhe, Arg or DArg

Z is 0 to 3 amino acid residues; and

B is the residue of a molecule comprising two carboxylic groups or is absent.

In a preferred embodiment, one of R is the side chain of Arg and Y is hydrogen or Y is the side chain of Arg and R is hydrogen. In another preferred embodiment, one of W₁ and W₂ is Phe and the other is absent. In another preferred embodiment, Z is absent or is selected from -Gln-Gly-Ser- and -Ala-Gly-Ser-. In an even more preferred embodiment, B is a residue of a dicarboxylicacid molecule preferably a residue of succinic acid, glutaric acid, phtalic acid, or pimelic acid. In an additional embodiment, n is 1.

Specifically preferred compounds of the present invention include those where a) Y is hydrogen; n is 1; W₁ is absent; W₂ is Phe; Z is -Gln-Gly-Ser-; n is 1; R is the side chain of Arg; and B is —CO—(CH₂)₅—CO—; where b) Y is the side chain of Arg; n is 1; W₁ is absent; W₂ is Phe; Z is absent; m is 5; and R is hydrogen; where c) Y is hydrogen; n is 1; the W₁ is Phe; W₂ is absent; Z is -Gln-Gly-Ser-; n is 1; R is the side chain of Arg; and B is a residue of a dicarboxylic acid; and where d) Y is hydrogen; n is 1; W₁ is Phe; W₂ is absent; Z is -Gln-Gly-Ser-; X is the side chain of Arg and B is a residue of a dicarboxylic acid.

According to specific embodiments the backbone cyclic compounds are of General Formula III (SEQ ID NO:1):

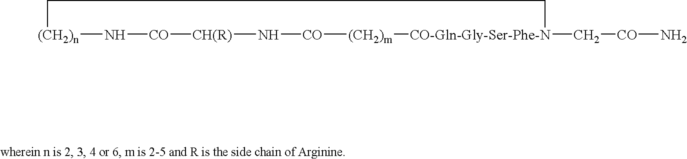

According to other specific embodiments the CD4 mimetics are selected from the group consisting of Formulae IV-XII:

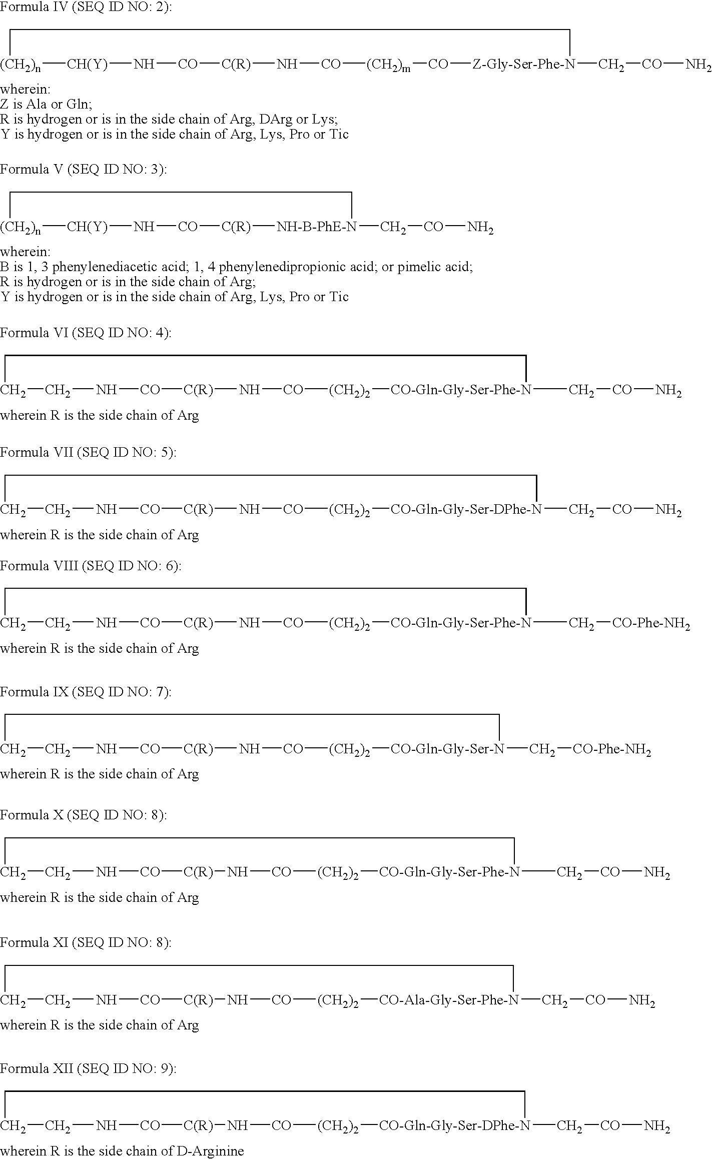

According to yet other specific embodiments the CD4 mimetics are of General Formula XIII:

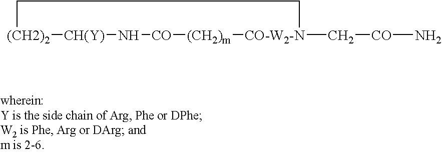

Specific preferred embodiments according to the present invention are selected from the group consisting of:

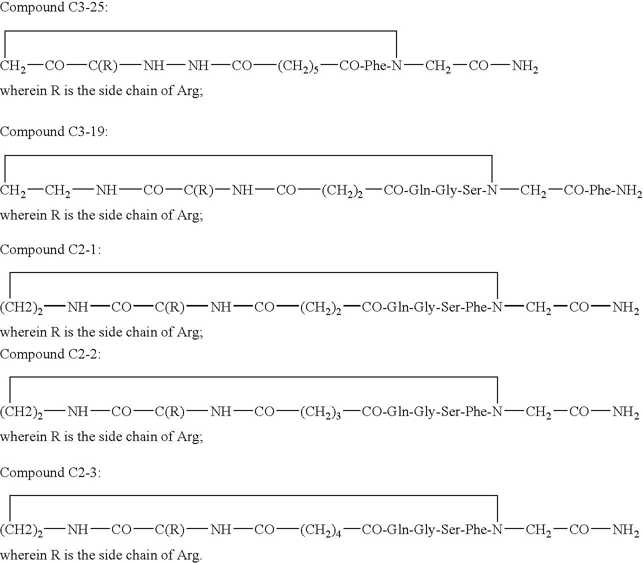

The pharmaceutical compositions comprising pharmacologically active molecule, preferably a backbone-cyclized that mimics the active site of the human CD4 protein, and a pharmaceutically acceptable carrier or diluent represent another embodiment of the invention, as do the methods for the prevention and treatment of viral infections and particularly HIV infections using such compositions.

The present invention further provides a method of treating a subject with HIV, comprising administering to the subject a backbone cyclized peptide analog that mimics the human CD4 protein binding site to gp120. More specifically, the peptide sequence comprises at least one aromatic residue and at least one positively charged residue. According to specific embodiments the aromatic residue is Phe or DPhe and the positively charged residue is Arg or DArg.

### BRIEF DESCRIPTION OF THE DRAWINGS

FIG. 1 shows the type II′ β-turn of the CD4 active site. The hydrogen bond between Phe43 backbone oxygen and Gln40 backbone nitrogen is illustrated.

FIG. 2 shows the results of CD4+ human cells infection inhibition by C2 peptides. Peptides were assayed at 100 μg/ml. One control used the 13B8.2 antibody, the other two controls are with and without virus (HIV+, HIV−). Each value is the means of 3 separate assays. Inhibition percent was determined according to β-galactosidase inhibition levels.

FIG. 3 shows the infection inhibition curves of peptides C2-1, C2-2 and C2-3 used to obtain their IC₅₀ values. C2-1 reached 50% inhibition at 33 μM. Peptides C2-2 and C2-3 have average IC₅₀ value of 81±2 μM.

FIG. 4 shows the effect of ring size on the inhibition of infection of CD4+ human cells by HIV-1 in the C2 peptides. The smaller the ring, the stronger is the inhibition.

FIG. 5 shows the effect of the length of the alkyl arm of the building unit (n in Formula II) and the type of dicarboxylic acid (m in Formula II), on the infection inhibition activity of the C2 peptides. Best inhibition is achieved with building unit having alkyl arm length of n7-2.

FIG. 6 shows the results of CD4+ human cells infection inhibition by the C3 peptides. Controls are with and without virus (HIV+, HIV−). Peptides were assayed at 100 μg/ml. Each value is the mean of 3 separate assays. Inhibition percent was determined according to β-galactosidase inhibition levels. The most active peptide is C3-25.

### DETAILED DESCRIPTION OF INVENTION

The present invention is directed to peptide analogs which mimic the non-continuous site of the CD4 protein and as a result inhibit the binding of the virus containing the gp120 molecule to the patient's cells. The invention further relates to compositions and methods for preventing or treating a retroviral infection, more particularly an HIV infection, and even more particularly an HIV-1 infection. The compositions and methods involve molecules that mimic the active site of the human CD4 protein, specifically the compositions and methods involves backbone cyclic peptide analogs which were designed and synthesized using a peptido/proteino-mimetic approach and further optimized to possess improved activity, permeability and stability properties.

The present invention provides backbone cyclization proteinomimetics which are functional mimetics of the binding site of CD4 protein responsible for binding to the viral gp120. These backbone cyclic analogs which may serve as leads for anti viral and anti HIV therapeutics, are according to General Formula II:

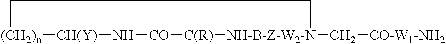

wherein:

Y is hydrogen or is the side chain of Arg, DArg, Lys, DLys, Phe, DPhe, Tic, DTic, Pro, or DPro;

n is 1 to 5;

R is the side chain of Arg, Lys, Phe, DArg, DLys or DPhe;

W₁ is absent or is Phe;

W₂ is absent or is Phe, DPhe, Arg or DArg

Z is 0 to 3 amino acid residues; and

B is the residue of a molecule comprising two carboxylic groups or is absent.

A set of backbone cyclic peptides according to one embodiment of the present invention, contains peptide analogs which bear the same parent sequence but differ in their ring size and thus also in their conformational ensemble. These compounds are represented by Formula III (SEQ ID: NO: 1):

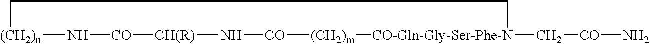

wherein n is 2, 3, 4 or 6, m is 2-5 and R is the side chain of Arginine.

Another set of peptide analogs, designed based on the structure of the most active backbone cyclic analog of the first set represents additional embodiments of the present invention. The compounds of this set of analogs are illustrated by Formulae IV-XII:

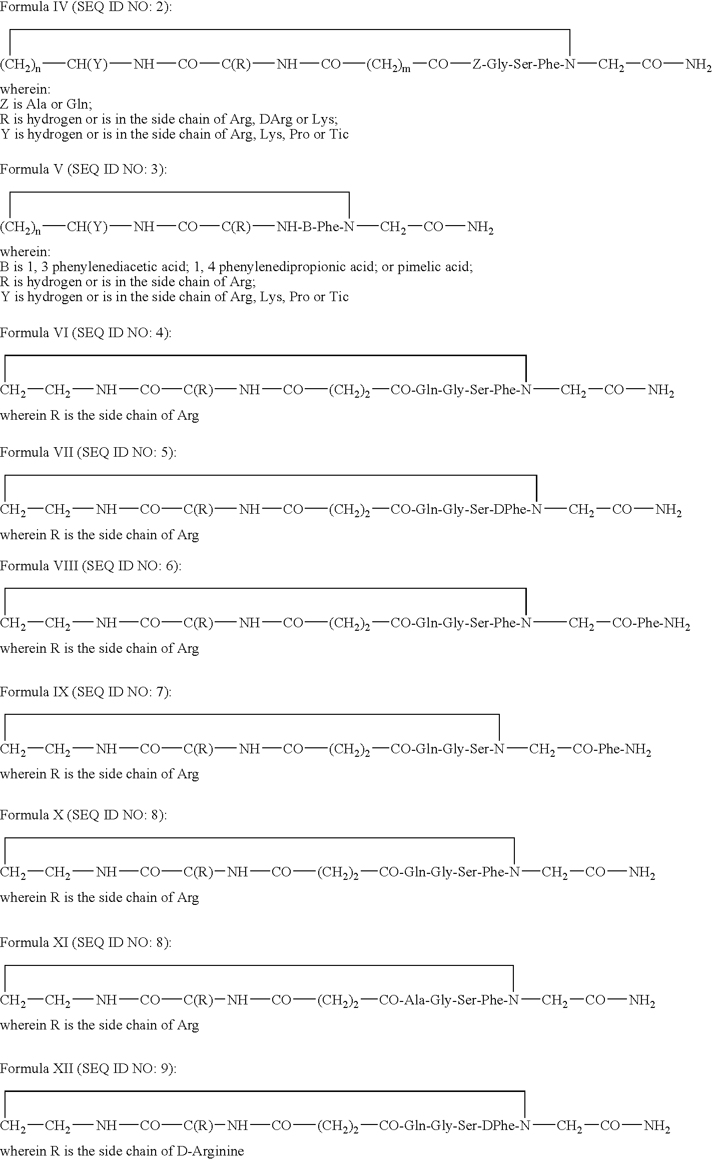

According to another embodiment, additional backbone cyclized peptide analogs each comprises one aromatic side chain and one positively charged side chain, are represented by Formula XIII:

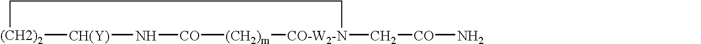

wherein:

Y is the side chain of Arg, Phe or DPhe;

W₂ is Phe, Arg or DArg; and

m is 2-6.

The present invention provides backbone cyclic proteinomimetics that are functional mimetics of an active region that bears a defined secondary structure within a protein. Specifically, the present invention provides backbone cyclic peptides, whose amino acid sequences correspond to the binding site of the CD4 protein, which are able to inhibit HIV-1 infection to human culture cells.

Conformational restriction renders the backbone cyclic CD4 mimetic less flexible and probably more selective than the linear peptides. In addition, the backbone cyclic peptides are resistant to proteolysis, a fact that should potentiate their metabolic stability. Being metabolically stable makes such backbone cyclic peptides attractive candidates for therapeutic applications. Evidently, such an approach was essential in the current case when the CD4 binding site is composed of discontinuous amino acid residues.

The present invention is also directed to a method of treating a subject with HIV, comprising administering to the subject a compound that mimics the human CD4 binding site. Such a compound can be a cyclic peptide. More specifically, the peptide includes Arg and Phe residues, and the ring portion of the cyclic peptide includes alkyls. An Arg residue may be replaced by another basic amino acid residue such as lysine or histidine, or non-naturally occurring residues, as described below. A Phe residue may be replaced by another aromatic amino acid residue such as tyrosine or tryptophan, or hydrophobic residue such as valine, leucine, etc., or non-naturally occurring residues, as described below.

### Determination of Anti HIV Activity

The above-described compounds of the present invention have anti-HIV activity. Such activity can be determined, for example, by the infection inhibition and β-galactosidase assays described in Example 1 below. Such activity can also be determined by measuring the concentration required to reduce the cytopathic effect of the virus, as described by Santosh et al., Bioorg. Med. & Chem. Lett., 10: 2505-08 (2000).

Such activity can also be determined using a plaque formation assay, and measuring the dose-dependent decrease in plaques, as described by Luedtke et al., Chembiochem, 3: 766-771 (2002); and Richman et al., Curr. Prot. Immun., pp. 1-21 (Wiley & Sons 1993). Dose-dependent activity can also be determined by measuring the decrease in HIV-1 p24 expression using ELISA. See Luedtke et al. and Richman et al., supra.

In addition, high-throughput screening assays can be performed to identify, for example, potential inhibition of HIV integration into the host cell chromosome. See Vandergraaf et al., Antimicrobial Agents and Chemotherapy, 45: 2510-16 (2001).

Conditions which may be prevented or treated with the compounds of the present invention include all conditions associated with HIV and other pathogenic retroviruses, including AIDS, AIDS-related complex (ARC), progressive generalized lymphadenopathy (PGL), as well as chronic CNS diseases caused by retroviruses, such as HIV mediated dementia and multiple sclerosis.

The compounds of the present invention can therefore be used as medicines against the above-mentioned conditions. The use comprises administering to HIV-infected subjects, or subjects at risk for HIV infection, an amount effective to combat the conditions associated with HIV and other pathogenic retroviruses, including HIV-1.

### Chemistry

The term “amino acid” refers to compounds which have an amino terminus and carboxy terminus, preferably in a 1,2-1,3-, or 1,4-substitution pattern on a carbon backbone. α-amino acids are most preferred and include the 20 natural amino acids (which are L-amino acids except for glycine), which are found in proteins, the corresponding D-amino acids, the biosynthetically available amino acids which are not found in proteins (e.g., 4-hydroxy-proline, 5-hydroxy-lysine, citrulline, ornithine, canavanine, djenkolic acid, .beta.-cyanolanine), and synthetically derived α-amino acids, such as amino-isobutyric acid, norleucine, norvaline, homocysteine and homoserine. β-Alanine and γ-amino butyric acid are examples of 1,3 and 1,4-amino acids, and many others are well known to the art, such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, both of which are incorporated herein by reference. Amino acids and amino acid analogs can be purchased commercially (Sigma Chemical Co.; Advanced Chemtech) or synthesized using methods known in the art. Statine-like isosteres (a dipeptide comprising two amino acids wherein the CONH linkage is replaced by a CHOH), hydroxyethylene isosteres (a dipeptide comprising two amino acids wherein the CONH linkage is replaced by a CHOHCH₂), reduced amide isosteres (a dipeptide comprising two amino acids wherein the CONH linkage is replaced by a CH₂ NH linkage) and thioamide isosteres (a dipeptide comprising two amino acids wherein the CONH linkage is replaced by a CSNH linkage) are also useful residues for this invention.

The amino acids used in this invention are those which are available commercially or are available by routine synthetic methods. Certain residues may require special methods for incorporation into the peptide, and either sequential, divergent and convergent synthetic approaches to the peptide sequence are useful in this invention. Natural coded amino acids and their derivatives are represented by three-letter codes according to IUPAC conventions. When there is no indication, the L isomer was used. The D isomers are indicated by “D” before the residue abbreviation.

List of Non-coded amino acids: Abu refers to 2-aminobutyric acid, Aib refers to 2-amino-isobutyric acid, Cha refers to cyclohexylalanine, Hcys refer to homocysteine, Hyp refers to S-trans-4-hydroxyproline, 1Nal refers to 1-naphtylalanine, 2NaI refers to 2-naphtylalanine, Nva refers to norvaline, Oic refers to octahydroindolecarboxylic acid, Phg refers to phenylglycine, pClPhe refers to p-chloro-phenylalanine, pFPhe refers to p-fluoro-phenylalanine, pNO2Phe refers to p-nitro-phenylalanine, Thi refers to thienylalanine.

Conservative substitutions of amino acids as known to those skilled in the art are within the scope of the present invention. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

As used herein “peptide” indicates a sequence of amino acids linked by peptide bonds. The peptides according to the present invention comprise a sequence of 3 to 12 amino acid residues, preferably 3 to 6 residues. A peptide analog according to the present invention may optionally comprise at least one bond, which is an amide-replacement bond such as urea bond, carbamate bond, sulfonamide bond, hydrazine bond, or any other covalent bond.

Salts and esters of the peptides of the invention are encompassed within the scope of the invention. Salts of the peptides of the invention are physiologically acceptable organic and inorganic salts. Functional derivatives of the peptides of the invention covers derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide and do not confer toxic properties on compositions containing it. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

The term “analog” indicates a molecule, which has the amino acid sequence according to the invention except for one or more amino acid changes. The design of appropriate “analogs” may be computer assisted. A peptide analog according to the present invention may optionally comprise at least one bond which is an amide-replacement bond such as urea bond, carbamate bond, sulfonamide bond, hydrazine bond, or any other covalent bond.

The term “peptidomimetic” means that a peptide according to the invention is modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with other covalent bond. A peptidomimetic according to the present invention may optionally comprises at least one bond which is an amide-replacement bond such as urea bond, carbamate bond, sulfonamide bond, hydrazine bond, or any other covalent bond. The term “proteinomimetic” refers to a peptidomimetic which is designed based on a non-continuous sequence of a protein site or region, namely mimic the conformation of residues which are adjacent in space but not necessarily contiguous in the protein sequence. The design of appropriate “peptidomimetic” or “proteinomimetic” may be computer assisted.

### Cyclic Peptides and Backbone Cyclization

Cyclization of peptides has been shown to be a useful approach in developing diagnostically and therapeutically useful peptidic and peptidomimetic agents. Cyclization of peptides reduces the conformational freedom of these flexible, linear molecules, and often results in higher receptor binding affinities by reducing unfavorable entropic effects. Because of the more constrained structural framework, these agents are more selective in their affinity to specific receptor cavities. By the same reasoning, structurally constrained cyclic peptides confer greater stability against the action of proteolytic enzymes (Humphrey, et al., Chem. Rev., 2243-2266 (1997)).

Methods for cyclization can be classified into the so-called “backbone to backbone” cyclization by the formation of the amide bond between the N-terminal and the C-terminal amino acid residues, and cyclizations involving the side chains of individual amino acids. The latter method includes the formation of disulfide bridges between two ω-thio amino acid residues (cysteine, homocysteine), the formation of lactam bridges between glutamic/aspartic acid and lysine residues, the formation of lactone or thiolactone bridges between amino acid residues containing carboxyl, hydroxyl or mercapto functional groups, the formation of thioether or ether bridges between the amino acids containing hydroxyl or mercapto functional groups and other special methods. In this review, recently developed general methods to effectively construct some of the aforementioned cyclic peptide derivatives will be covered along with some ingenious approaches to form specific cyclic peptide analogues. A notable recent review should also serve as a useful resource on a variety of peptide cyclization methodologies (Lambert, et al., J. Chem. Soc. Perkin Trans., 1: 471-484 (2001)).

Backbone cyclized analogs are peptide analogs cyclized via bridging groups attached to the alpha nitrogens or alpha carbonyl of amino acids that permit novel non-peptidic linkages. In general, the procedures utilized to construct such peptide analogs from their building units rely on the known principles of peptide synthesis; most conveniently, the procedures can be performed according to the known principles of solid phase peptide synthesis. During solid phase synthesis of a backbone cyclized peptide the protected building unit is coupled to the N-terminus of the peptide chain or to the peptide resin in a similar procedure to the coupling of other amino acids. After completion of the peptide assembly, the protective group is removed from the building unit's functional group and the cyclization is accomplished by coupling the building unit's functional group and a second functional group selected from a second building unit, a side chain of an amino acid residue of the peptide sequence, and a N-terminal amino acid residue.

As used herein the term “backbone cyclic peptide” or “backbone cyclic analog” refers to a sequence of amino acid residues wherein at least one nitrogen or carbon of the peptide backbone is joined to a moiety selected from another such nitrogen or carbon, to a side chain or to one of the termini of the peptide. According to specific embodiment of the present invention the peptide sequence is of 3 to 12 amino acids that incorporates at least one building unit, said building unit containing one nitrogen atom of the peptide backbone connected to a bridging group comprising an amide, thioether, thioester, disulfide, urea, carbamate, or sulfonamide, wherein at least one building unit is connected via said bridging group to form a cyclic structure with a moiety selected from the group consisting of a second building unit, the side chain of an amino acid residue of the sequence or a terminal amino acid residue. Furthermore, one or more of the peptide bonds of the sequence may be reduced or substituted by a non-peptidic linkage.

A “building unit” (BU) indicates an Na or Ca derivatized amino acid. An Na derivatized amino acid is represented by the General Formula XIV:

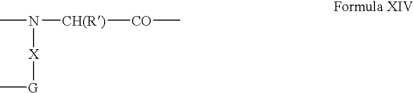

wherein X is a spacer group selected from the group consisting of alkylene, substituted alkylene, arylene, cycloalkylene and substituted cycloalkylene; R′ is an amino acid side chain, optionally bound with a specific protecting group; and G is a functional group selected from the group consisting of amines, thiols, alcohols, carboxylic acids, sulfonates, esters, and alkyl halides; which is incorporated into the peptide sequence and subsequently selectively cyclized via the functional group G with one of the side chains of the amino acids in said peptide sequence, with one of the peptide terminals, or with another ω-functionalized amino acid derivative.

The present invention is exemplified by using Nα derivatized Glycine of the General Formula XV:

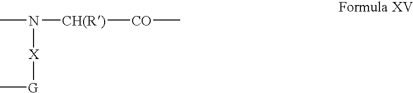

wherein X is alkylene, R′ is a hydrogen; and G is amine; which is incorporated into the peptide sequence and subsequently selectively cyclized via the functional group G with a carboxylic group attached to the N-terminus of said peptide sequence. According to specific embodiments of the present invention X in Formula XV is an alkylene substituted with a side chain of an amino acid. According to more specific embodiments X is selected form the group consisting of CH-side chain of Arg, CH-side chain of Lys, CH-side chain of Phe, and CH-side chain of Tic, wherein Tic refers to Tetrahydroisoquinoline-3-carboxylic acid residue.

The building units in the present invention are depicted in their chemical structure as part of the peptide sequence or are abbreviated by the three letter code of the corresponding modified amino acid preceded by the type of reactive group (N for amine, C for carboxyl). For example, N-Gly describes a modified Gly residue with an amine reactive group thus, according to the present invention, N-Gly within a sequence of a backbone cyclized peptide is equal to NH—(CH₂)n—N—CH₂—CO—NH₂

The methodology for producing the building units is described in international patent applications published as WO 95/33765 and WO 98/04583 and in U.S. Pat. Nos. 5,770,687 and 5,883,293 all of which are expressly incorporated herein by reference thereto as if set forth herein in their entirety.

The term “bridging group” according to the present invention refers to a chemical linker or spacer connecting a nitrogen atom of the peptide backbone to a second building unit, to a side chain of an amino acid residue of the sequence or to a terminal amino acid residue. According to some embodiments the chemical linker or spacer group is presented by the general Formula (I):

—(CH)ₙ—(CH)Y-M-A-B- Formula I

wherein n is an integer for 1 to 8; M is selected from the group consisting of a disulfide, amide, thioether, thioester, imine, ether, or alkene bridge; Y is hydrogen or an amino acid side chain; A is (CH₂)ₘ wherein m is an integer for 1 to 8, or C(R)—NH wherein R is an amino acid side chain; and B is absent or is the residue of a molecule comprising two carboxylic groups. Non-limiting examples of B according to the present invention are succinic acid residue and phthalic acid residue.

Backbone cyclized peptides according to the present invention may be synthesized using any method known in the art, including peptidomimetic methodologies. These methods include solid phase as well as solution phase synthesis methods. Non-limiting examples for these methods are described hereby. Other methods known in the art to prepare compounds like those of the present invention can be used and are comprised in the scope of the present invention.

The methods for design and synthesis of backbone cyclized analogs according to the present invention are disclosed in U.S. Pat. Nos. 5,811,392; 5,874,529; 5,883,293; 6,051,554; 6,117,974; 6,265,375, 6,355,613, 6,407,059, 6,512,092 and international applications WO 95/33765; WO 97/09344; WO 98/04583; WO 99/31121; WO 99/65508; WO 00/02898; WO 00/65467 and WO 02/062819. All of these methods are incorporated herein in their entirety, by reference.

The most striking advantages of backbone cyclization are: 1) cyclization of the peptide sequence is achieved without compromising any of the side chains of the peptide thereby decreasing the chances of sacrificing functional groups essential for biological recognition (e.g. binding to specific receptors), and function; 2) optimization of the peptide conformation is achieved by allowing permutation of the bridge length, and bond type (e.g., amide, disulfide, thioether, thioester, urea, carbamate, or sulfonamide, etc.), bond direction, and bond position in the ring; 3) when applied to cyclization of linear peptides of known activity, the bridge can be designed in such a way as to minimize interaction with the active region of the peptide and its cognate receptor. This decreases the chances of the cyclization arm interfering with recognition and function.

The principles of the “backbone cyclic peptidomimetic” and “backbone cyclic proteinomimetic” approaches are based on the following steps: (i) elucidation of the active residues in the target protein (ii) design and modeling of an ensemble of prototypic backbone cyclic peptides that encompass the active residues and their conformation resemble that of the parent protein (iii) cycloscan of each backbone cyclic prototype until a lead compound is discovered (iv) structural analysis of the best lead and (v) optimization through iteration.

“Cycloscan” is a selection method based on conformationally constrained backbone cyclic peptide libraries that allows rapid detection of the most active backbone cyclic peptide derived from a given sequence as disclosed in WO 97/09344. The teachings of this disclosure are incorporated herein in their entirety by way of reference. The diversity of cycloscan, which includes modes of backbone cyclization, ring position, ring size and ring chemistry allows the generation of a large number of sequentially biased peptides that differ solely by their conformation in a gradual discrete manner.

### Pharmacology

The compounds of the present invention can be formulated into various pharmaceutical forms for purposes of administration. For example, a compound of the invention, or its salt form, a N-oxide form or a stereochemically isomeric form, can be combined with a pharmaceutically acceptable carrier. Such a carrier can depend on the route of administration, such as oral, rectal, percutaneous or parenteral injection.

For example, in preparing the compositions in oral dosage form, media such as water, glycols, oils, alcohols can be used in liquid preparations such as suspensions, syrups, elixirs, and solutions. Alternatively, solid carriers such as starches, sugars, kaolin, lubricants, binders, disintegrating agents can be used, for example, in powders, pills, capsules or tablets.

For parenteral compositions, the carrier can comprise sterile water. Other ingredients may be included to aid in solubility. Injectable solutions can be prepared where the carrier includes a saline solution, glucose solution or mixture of both.

Injectable suspensions can also be prepared. In addition, solid preparations that are converted to liquid form shortly before use can be made. For percutaneous administration, the carrier can include a penetration enhancing agent or a wetting agent.

It can be advantageous to formulate the compositions of the invention in dosage unit form for ease of administration and uniformity of dosage. “Dosage unit form” refers to physically discrete units suitable as unitary dosages, each unit containing a pre-determined quantity of active ingredient calculated to produce the desired therapeutic effect in association with the chosen carrier.

Apart from other considerations, the fact that the novel active ingredients of the invention are peptides, peptide analogs or peptidomimetics, dictates that the formulation be suitable for delivery of these types of compounds. Although in general peptides are less suitable for oral administration due to susceptibility to digestion by gastric acids or intestinal enzymes. According to the present invention, novel methods of backbone cyclization are being used, in order to synthesize metabolically stable and oral bioavailable peptidomimetic analogs. The preferred route of administration of peptides of the invention is oral administration.

Other routes of administration are intra-articular, intravenous, intramuscular, subcutaneous, intradermal, or intrathecal.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

For buccal administration, the compositions may take the form of tablets or lozenges formulated in conventional manner.

For administration by inhalation, the variants for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., Curr. Opin. Chem. Biol. 5, 447, 2001). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate symptoms of a disease of the subject being treated. Determination of a therapeutically effective amount is well within the capability of those skilled in the art.

Toxicity and therapeutic efficacy of the peptides described herein can be determined by standard pharmaceutical procedures in cell cultures or experimental animals, e.g., by determining the IC₅₀ (the concentration which provides 50% inhibition) and the LD50 (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in “The Pharmacological Basis of Therapeutics”, Ch. 1 p. 1).

Those skilled in the treatment and prevention of HIV infection can determine the effective daily amount. Generally, an effective amount can be from 0.01 mg/kg to 50 mg/kg body weight and, more preferably, from 0.1 mg/kg to 10 mg/kg body weight. It may be appropriate to administer the required dose as two, three, four or more sub-doses at appropriate intervals during the day. Such sub-doses can be formulated as unit dosage forms, for instance, containing 1 to 1000 mg, more preferably 5 to 200 mg, of active ingredient per unit dosage form.

The precise dosage and frequency of administration depends on the particular compound of the invention being used, as well as the particular condition being treated, the severity of the condition, the age, weight, and general physical condition of the subject being treated, as well as other medication being taken by the subject, as is well known to those skilled in the art. It is also known that the effective daily amount can be lowered or increased depending on the response of the subject or the evaluation of the prescribing physician. Thus, the ranges mentioned above are only guidelines and are not intended to limit the scope of the use of the invention.

The combination of a compound of the invention with another anti-retroviral compound can be used.

The following examples are intended to illustrate but not limit the present invention.

### EXAMPLES

In order to design a small backbone cyclic peptidic molecule mimicking the gp120-binding site of CD4, the structure of this site was studied.

CD4 is a mostly extra-cellular co-receptor embedded in the T cell membrane by a trans-membranal domain, followed by a short intra-cellular domain. This protein is very important in proper function of the immune system, mainly in the binding of CD4+ T cells to antigen presenting cells.

The most important residue in the active site of CD4, i.e., the gp120-binding site, is CD4's Phe43. This residue is situated on a type II′ β-turn and its phenyl ring enters a hydrophobic pocket in gp120. This residue is responsible for 23% of the binding interactions between the two proteins, either by hydrophobic interactions of its phenyl ring or by both hydrophobic and hydrophilic interactions of its backbone atoms. It interacts with many gp120 residues: Glu370, Ile371, Asn425, Met426, Trp427, Gly473 and Asp368. Only the interaction with Ile371 is a classical hydrophobic one. There is also an aromatic stacking interaction of its phenyl ring with the carboxylate group of Glu370. Other interactions involve backbone atoms only. The second important residue is Arg59 of CD4. This residue forms a hydrogen bond with Asp368 of gp120. Residues Lys46, Lys35 and Lys29 are less important. Residues Asp368, Glu370 and Trp427, as well as the residues forming the hydrophobic pocket of gp120, were found to be conserved amongst various HIV strains. This shows their high importance in activity. A few point mutations were found to increase the binding affinity of the two proteins. Replacing Arg59 with a Lys residue tripled binding affinity, while replacing Gln40 or Asp63 by Ala doubles it.

Based on the above, backbone cyclic peptide libraries were designed and tested for their ability to inhibit the CD4-gp120 binding interaction.

### Materials and Methods

Organic synthesis reagents were purchased usually from Aldrich and Merck. Organic solvents were purchased from Frutarom. Protected and un-protected amino acids, coupling reagents, protecting groups and resins were purchased from Novabiochem and Bachem except for Alloc-Arg (Mts)-OH which was prepared as described herein. Ultra pure solvents for peptide synthesis and HPLC analysis were purchased from J. T. Baker. All solvents and reagents were used as is without further purification. TLC was performed on F₂₅₄ silica plates (Merck). Detection was performed by one of the three following methods: UV at 254 nm, 1% ninhydrin in methanol or iodine. Analytical HPLC was performed on Merck-Hitachi systems: 1. Model 665A with a LC-6200A gradient pump, L-4200 UV/Vis detector and 655A-40 autosampler. 2. Model LaChrom with a L-7100 pump, L-7200 autosampler, L-7400 UV/Vis detector and a D-7000 interface. Products were assayed at 215 and 220 nm. Mobile phase solvents were triple distilled water and acetonitrile with addition of 1% trifluoroacetic acid at 1 ml/min. Lichrospher (Merck) and Vydac RP-18 columns were used. Their dimentions: 25 cm long and 5 mm inner diameter. Semi-preparative HPLC separations were performed on a Merck-Hitachi 665A model equipped with a preparative pump (30 ml/min) and a high flow UV/is detector. Solvents and wavelengths were as for analytical HPLC at 4.5 ml/min for the semi-preparative separations. An RP-18 Vydac column 25 cm long and 10 mm in diameter was used. HNMR was performed on a Brucker AMX-300 system at 295° K. Peptides were prepared by the SMPS method in polypropylene bags shaken in polyethylene boxes. Shakers used were Labotron by Infors HT and Bigger Bill by Thermolyne.

### Preparation of Regular Amine and Carboxyl Backbone Cyclization Building Units.

The building units were synthesized by procedures described in Muller et al., J. Org. Chem., 62: 411-16 (1997).

Preparation of N^{α}-(Boc-amino acids)N,O-dimethyl hydroxamates (2)

To a solution of 0.055 mole N,O-dimethylhydroxylamine hydrochloride in 100 ml DMF were added 0.05 mole of 1, 0.055 mole PyBOP and 0.15 mole DIEA. Reaction mixture was left to stir at r.t. for 3 hours while maintaining pH at 9-10. Then 300 ml of EtOAc were added while stirring followed by 600 ml saturated NaHCO₃. The organic phase was washed with saturated NaHCO₃ (2×100 ml), water (2×100 ml), 1M KHSO₄ (2×100 ml) and water (2×100 ml), dried over Na₂SO₄ and evaporated to dryness. Product was left to dry in dessicator. TLC monitoring solvent system: EtOAc:PE (1:1). Products were obtained at 94-100% yields.

N^{α}-Boc-Arg(di-Z)N,O-dimethylhydroxanate (2a). HNMR (CDCl₃): 9.50, broad, 1H (NH); 7.33, m, 10H (Ar); 5.25, s, 2H (Ar—CH₂); 5.15, s, 2H (Ar—CH₂); 4.60, broad, 1H (Hα); 3.98, t, 2H(Hδδ′); 3.67, s, 3H(OCH₃); 3.12, s, 3H(NCH₃); 1.70, m, 4H(Hδβ′, Hγγ′); 1.40, s, 9H (Boc).

N^{α}-Boc-Lys(Z)N,O-dimethylhydroxamate (2b). HNMR (CDCl₃): 7.33, m, 5H (Ar); 5.10, s, 2H (Ar—CH₂); 4.67, s, 1H (Ha); 3.70, s, 3H(OCH₃); 3.20, s, 3H(NCH₃); 3.10, m, 2H (Hεε′); 1.82, m, 4H(Hββ′, Hδδ′); 1.67, m, 2H (Hγγ′); 1.43, s, 9H (Boc).

N^{α}-Boc-Pro-N,O-dimethylhydroxamate (2c). HNMR (CDCl₃): 4.60, broad, 1H (Hα); 3.71, s, 3H(OCH₃); 3.19, s, 3H(NCH₃); 1.88, m, 6H (Hββ′, Hγγ′, Hδδ′); 1.41, s, 9H (Boc).

N^{α}-Boc-Tic-N,O-dimethylhydroxamate (2d). HNMR (CDCl₃): 7.15, m, 4H (Ar); 4.82, t, 1H (Hα); 3.78, s, 3H(OCH₃); 3.15, s, 5H(NCH₃, H86′); 1.83, m, 2H(Hββ′); 1.45, s, 9H (Boc).

Preparation of N^{α}-(Boc-amino acids) aldehydes (3)

To a solution of 2 in 200 ml anhydrous THF in an ice bath and under Ar, was added portion-wise LiAlH₄ (2 eq.). When addition was over the ice bath was removed and reaction mixture was left to stir at r.t. for another hour. When reaction was over the ice bath was returned and 500 ml EtOAc was added portion-wise. Then 1200 ml 1M KHSO₄ was added and the reaction mixture was left to stir for another 30 min. Then the phases were separated and the organic phase was washed with 1M KHSO₄ (2×200 ml) and brine (2×200 ml), dried over Na₂SO₄ and evaporated to dryness. An oil was obtained. The product was kept at minus 8° C. under Ar. TLC monitoring solvent system: EtOAc:PE (1:1). Products were obtained at 55-93% yields.

N^{α}-Boc-Arg(di-Z) aldehyde (3a). HNMR (CDCl₃): 9.55, s, 1H(COH); 7.30, m, 10H (Ar); 5.15, s, 2H (Ar—CH₂); 1.72, m, 4H (Hββ′, Hγγ′); 1.37, s, 9H (Boc).

N^{α}-Boc-Lys(Z) aldehyde (3b). HNMR (CDCl₃): 9.55, s, 1H(COH); 7.33, m, 5H (Ar); 5.10, s, 2H (Ar—CH₂); 4.67, s, 1H (Hα); 3.12, m, 2H(Hδδ′); 1.82, m, 4H(Hββ′, Hδδ′); 1.67, m, 2H (Hγγ′); 1.43, s, 9H (Boc).

N^{α}-Boc-Pro aldehyde (3c). HNMR (CDCl₃): 9.45, s, 1H(COH); 3.50, m, 2H (Hδδ′); 1.79, m, 4H (Hββ′, H′γγ); 1.42, s, 9H (Boc).

N^{α}-Boc-Tic aldehyde (3d). HNMR (CDCl₃): 9.53, s, 1H(COH); 7.17, m, 4H (Ar); 4.67, s, 1H (Hα); 3.15, m, 2H(Hδδ′); 1.81, m, 2H(Hββ′); 1.43, s, 9H (Boc).

### Preparation of N⁶⁰-Alloc-Arg(Mts)-Oh

To a solution of 0.57 mole H-Arg(Mts)-OH in 43 ml 4N NaOH and 6 ml iPrOH cooled in an ice bath, was added portion-wise a solution of 10 ml allylchloroformate in 20 ml 4N NaOH and 2 ml iPrOH under vigorous stirring. When addition was over the reaction mixture was left to stir under cooling for another 40 min, after which the ice bath was removed and the reaction mixture was left to stir at r.t. o.n. pH was maintained at 11 at all times. When reaction was over, 45 ml water were added, the phases were separated and the hydrous phase was washed with PE (3×30 ml). Then the hydrous phase was cooled in an ice bath and gradually acidified by concentrated HCl to pH=1. A white sticky mush is obtained. The product was extracted to EtOAc (4×50 ml). The organic phase was dried over MgSO₄, evaporated to dryness and left to dry in the dessicator. The caramel solid obtained was dissolved in CHCl₃ (200 ml), washed with 1N HC1 (3×30 ml) and water (2×30 ml), dried over Na₂SO₄, evaporated to dryness and left to dry in the dessicator. A white precipitate was obtained. TLC monitoring solvent system: CHCl₃:MeOH (4:1). Yield: 73%. HNMR (CDCl₃): 6.89, s, 2H (Ar); 6.00, broad, 1H(NH); 5.82-5.95, split q, 1H(CH₂═CH—CH₂); 5.20, split d, 2H(CH₂═CH—CH₂); 4.55, d, 2H(CH₂═CH—CH₂); 2.62, s, 6H (Ar-oCH₃); 2.26, s, 2H (Ar-pCH₃).

### Peptide Synthesis—General Procedures.

Peptides were synthesized by a combination of Boc and Fmoc chemistries. α-Amines were protected by the Fmoc group while the side chains were protected by Boc chemistry protecting groups. When an amino acid was introduced as a linker between the building units, the Fmoc group was replaced on the growing peptide by a Dde group, prior to Boc deprotection from the building unit. All peptides were synthesized on MBHA resin using standard solid phase peptide synthesis procedures. All reactions were performed at r.t. in DMF, NMP or DCM. Each reaction was followed by resin washes to discard reaction reagents. Each coupling or cleavage step was followed by free amine standard Kaiser and chloranil detection assays in order to determine the step's success. Kaiser et al., Anal. Biochem., 34: 595-98 (1970); Christensen, T., Acta. Chem. Scand. B., 33: 763-66 (1979). Peptides were cleaved by standard HF or TMSOTf:TFA procedures.

### On-Resin Formation of Building Units.

The building units were formed by reductive alkylation of Gly residues which were coupled to the solid phase by aldehydes 3a-d. To bags containing the resin pre-loaded with Gly, was added a solution of 4 eq. of aldehyde 3 in NMP:MeOH (1:1) with 1% (v/v) AcOH. The peptides were shaken in this solution for 5 min. Then 4 eq. of NaBH₃CN were added and the peptides were left to shake in this reaction mixture for additional 3 hours. After completion the bags were washed with NMP:MeOH (1:1)+1% (v/v) AcOH (X1), DMF (X1), NMP (X2), DCM (X2), EtOH (X2) and finally DCM (X2).

### Example 1

### Design, Synthesis and Activity of the C2 Peptide Analogs

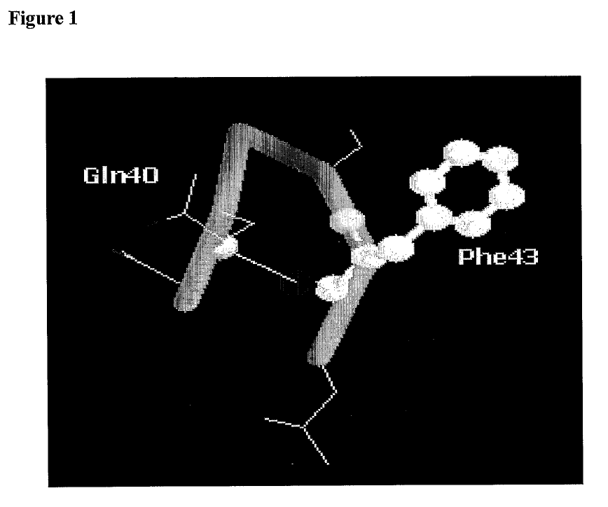

To mimic the structure and activity of CD4, asset of peptides was constructed based on a structural element already existing at the molecule's active site, specifically the type II′ β-turn containing the residue most important for the binding interaction, Phe43 as depicted in FIG. 1. This turn is held by a hydrogen bond formed between the backbone oxygen of Phe43 and the backbone nitrogen of Gln40. This turn was used as the major structural element of the scaffold, mimicking the mentioned hydrogen bond by cyclization of the building units. The Arg residue was inserted to improve the binding of the molecule to gp120 and, therefore, was inserted as a bridging residue between the building units in the ring. This location enabled keeping the native sequence intact, thus increasing the chances of obtaining the native turn. It also facilitated the scanning of the optimal positioning of Arg relative to the Phe residue without causing any synthetic problems. Hence, the compounds synthesized are represented by Formula III (SEQ ID NO:1):

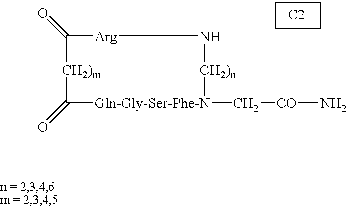

### Synthesis of the C2 Set of Backbone Cyclic Peptides

Peptides were synthesized on MBHA resin, in order to introduce an amide group at the carboxi-terminus, so as to form peptides resembling a peptidic segment of a protein. The peptides were synthesized using the “Tea Bag” method often used in our lab, according to well-established peptide synthesis protocols. Cyclization was performed in a bi-step manner, at each step one ring was closed; first a closure of the amide was performed, using a standard coupling reaction, then closure of the disulfide ring with iodine.

The peptides were cleaved off the solid phase with HF, purified by preparative HPLC using an RP-C18 preparative column in a water:acetonitrile gradient (programs 1, 4, 5) and characterized by MS. Their purity was determined by analytical HPLC on an RP-C18 analytical column in similar gradients, as shown in Table 1 below.

TABLE 1
Synthesis results, purity and characterization of the C2 peptides.

| | | | Purity^{a} | Calc. | Found | Net Wt. | |
|---|---|---|---|---|---|---|---|
| Peptide | n | m | (%) | MW^{b} | MW^{b} | (mg) | Yield (%) |
| C2-1 | 2 | 2 | 90 | 774.8 | 775.9 | 12.17 | 26.6 |
| C2-2 | 2 | 3 | 98 | 788.9 | 789.3 | 10.12 | 21.7 |
| C2-3 | 2 | 4 | 99 | 802.9 | 804 | 8.41 | 17.8 |
| C2-4 | 2 | 5 | 97 | 816.9 | 817.9 | 5.99 | 13.5 |
| C2-5^{c} | 3 | 2 | — | — | — | — | — |
| C2-6 | 3 | 3 | 98 | 802.9 | 803.4 | 9.38 | 19.5 |
| C2-7 | 3 | 4 | 98 | 816.9 | 817.4 | 6.31 | 13.7 |
| C2-8 | 3 | 5 | 96 | 831 | 831.4 | 5.44 | 11.9 |
| C2-9 | 4 | 2 | 47 | 802.9 | 804 | 9.83 | 19.6 |
| C2-10 | 4 | 3 | 94 | 816.9 | 817.4 | 8.69 | 18.1 |
| C2-11 | 4 | 4 | 92 | 831 | 832 | 6.42 | 12.4 |
| C2-12 | 4 | 5 | 98 | 845 | 845.9 | 6.21 | 10.6 |
| C2-13 | 6 | 2 | 58 | 831 | 832 | 7.97 | 17.3 |
| C2-14 | 6 | 3 | 97 | 845 | 845.9 | 8.48 | 14.9 |
| C2-15 | 6 | 4 | 98 | 859 | 859.9 | 6.11 | 11.6 |
| C2-16 | 6 | 5 | 98 | 873 | 873.9 | 5.83 | 11.2 |

^{a}Purity was determined by analytical HPLC.
^{b}MW in g/mole.
^{c}Synthesis not completed due to technical problems.

In order to avoid cleavage of the Trt protecting group from the Gln residue prior to the completion of the synthesis and cleavage off the solid support, an Alloc group was used to protect the N^{α} of the Arg residue. This protecting group is cleaved in mild conditions (see materials and methods chapter), keeping the Trt group intact, thus eliminating the possibility of obtaining undesired side reactions on the Gln side chain.

Biological Activity Results of C2 peptide analogs Cell Infection Inhibition Assay The assay consisted of CD4 expressing Hela P4 cells containing the α-galactosidase reporter gene placed downstream the HIV-LTR promotor. These cells, were seeded 24 hours prior to viral infection at a density of 5×10⁵ per cell in 24 well culture plates (TPP model by Beyneix). 50 μL of virus solution prepared from CEM infected cells' supernatant, were incubated for 1 hour with the assayed peptides at the required concentrations, at 4° C. 10 μg of anti CD4 monoclonal antibodies 13B8.2 or Leu3A were used as control. After incubation the virus solution was diluted to a total volume of 1 ml and was added to the Hela P4 cells. The cells were incubated at these conditions for 3 days after which their infection rate was assayed according to the α-galactosidase activity in the cells extract.

The cells were washed well, harvested and disintegrated in a buffer containing 60 mM Na₂HPO₄, 40 mM NaH₂PO₄, 10 mM KCl, 1 mM MgSO₄, 50 mM β-mercaptoethanol, 2.5 mM EDTA, 0.125% NP40, 0.125% triton, 20% glycerol, 0.2 mM PMSF and 100 U/ml approtinin. The cells extract was cleaned by centrifuge at 4° C. for 15 min at 13,000 rpm. β-galactosidase activity was determined by incubation of 150 μL of total cell extract at 37° C. for 2 hours in the presence of 6 mM ONPG (O-Nitrophenyl-β-D-galactopyranoside) in a buffer containing 80 mM Na₂HPO₄, 1 mM β-mercaptoethanol and 10 mMMgCl₂, followed by absorption measurement at 410 nm. The β-galactosidase activity was normalized according to the total protein quantity in the assay.

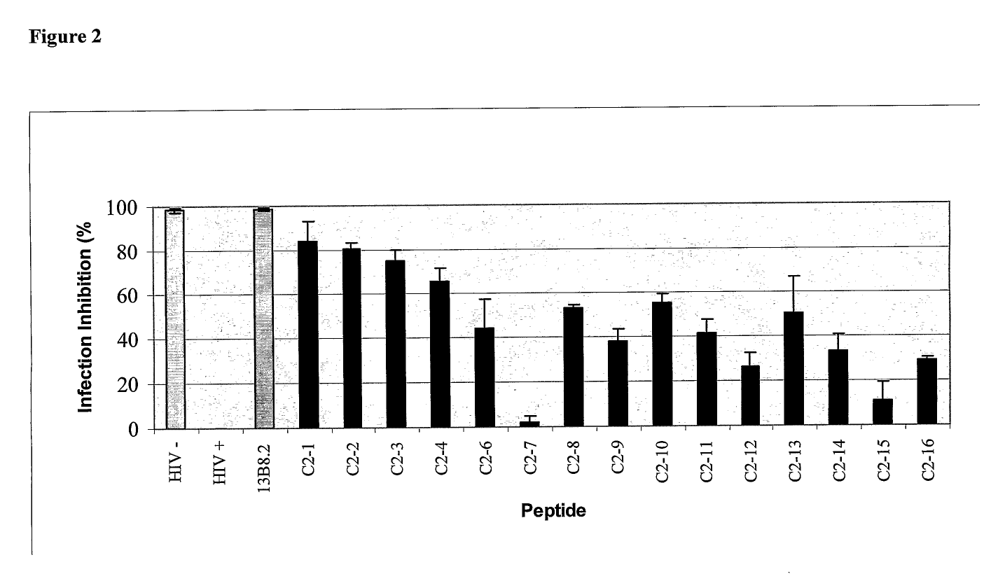

Results of this assay of the C2 peptides are shown in FIG. 2. As shown, peptides C2-1, C2-2 and C2-3 are the most active.

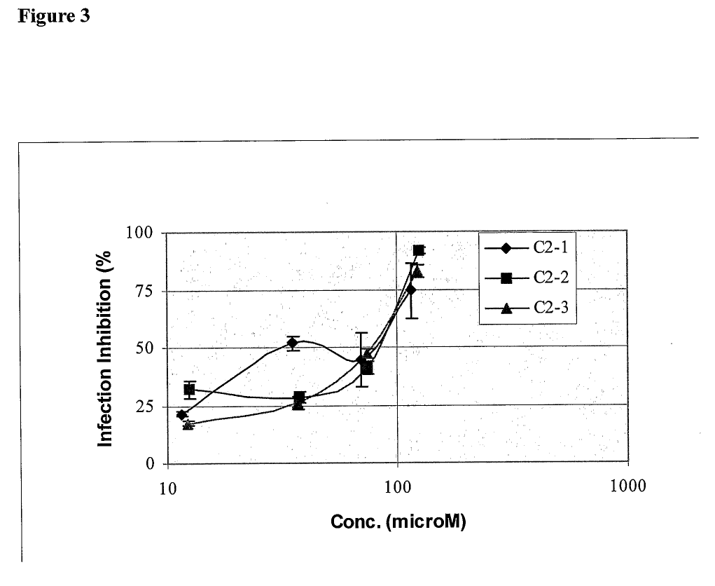

In order to better determine their activity level, from which a lead peptide could be selected, peptides C2-1, C2-2 and C2-3 were assayed in several concentrations so as to obtain their IC₅₀ values. The results of this assay as described in FIG. 3, show that indeed peptide C2-1 is most active, with an IC₅₀ value of 33 μM. This activity is already in the range of many lead compounds in pharmaceutical research. Peptides C2-2 and C2-3 act very similarly and their average IC₅₀ value is 81±2 μM.

Moreover, these peptides possess a distinct advantage over others. Specifically, C2-1, as well as other peptides in this set, is a small backbone cyclic peptide (7 amino acids and amino-acid equivalents only). This gives it immunological and pharmacological advantages over longer peptides.

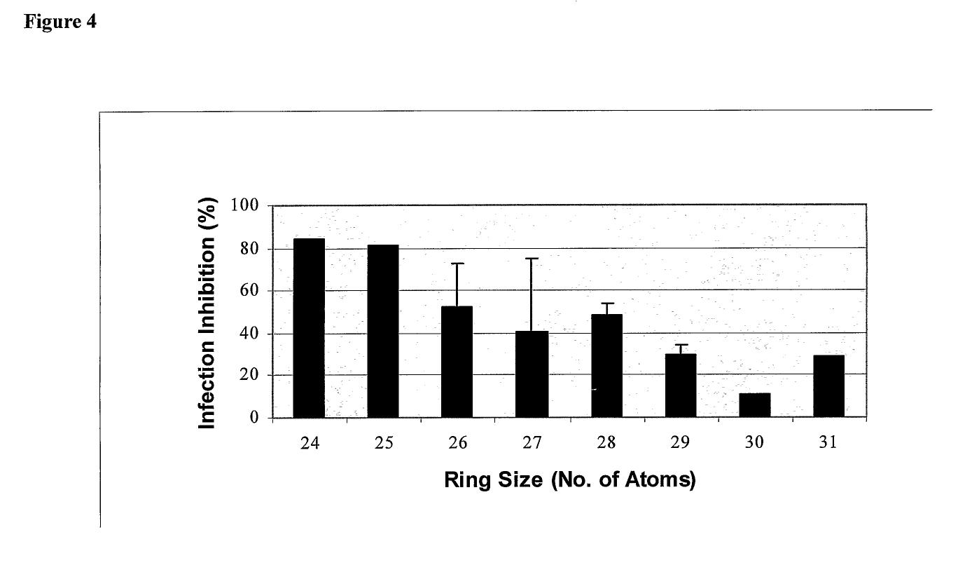

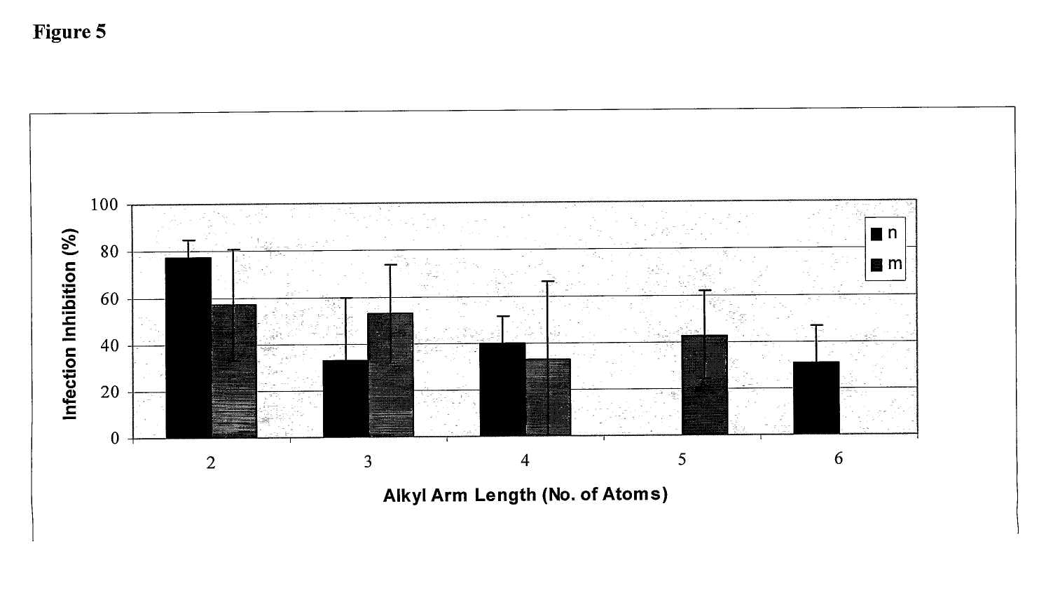

The activity of the C2 peptides was analyzed with respect to their structural elements, specifically, ring size and alkyl arm length. Generally, as shown in FIGS. 4 and 5, the larger the ring the less active the peptide. Moreover, the alkyl arm of the building unit (n) is more influential regarding the inhibitory activity of the peptides compared with the dicarboxylic acid arm (m). When n=2 the activity is much higher than at all other lengths (darker columns) while there is no significant difference in the peptides activities regarding different m values (lighter columns).

Based on the results above, it was concluded that it is important for the distance between Arg and Phe (via the building unit) to be minimal. The size of the other part of the ring (including the dicarboxylic acid) is less important. Based on these conclusions another set of peptides, C3 was designed and synthesized.

### Example 2

### Design, Synthesis and Activity of the C3 Peptides

### Design

This set of peptides was designed based on the lead peptide C2-1. In this set of peptides the influence of various factors on the inhibitory activity of the peptide was examined as follows:

A. Changing Arg with Lys and Gln with Ala. These mutations were found to increase activity in the native protein. (Peptides C3-1, 2, 3);

B. Shortening the alkyl arm of the building unit, because activity increases the shorter the distance is between Arg and Phe. (Peptides C3-4, 5, 6, 7);

C. Insertion of a steric hindrance element on the alkyl arm of the building unit. This can lead to a better constraining of conformation, and given that it is close to the native conformation this could increase the peptide's activity. (Peptides C3-8, 9, 10, 11, 12, 13, 14, 15);

D. Using D-Phe and D-Arg. (Peptides C3-16, 17);

E. Positioning of the Phe residue inside and outside the ring, increasing the conformational freedom of Phe;

F. Changing of the direction of the peptidic bonds in the ring. This involves the bonds between the building unit, the Arg residue and the acid contacting it to Gln. (Peptide C3-26);

G. Deletion of the amino acids at the far side of the ring (residues 40-42 in the native sequence). This was examined since there was no great importance to the size of that part of the ring. Conformational changes in this part are of less importance to activity and therefore they may play only a structural role that is limited to the backbone atoms. Thus changing this part of the molecule with a simpler chain, reducing the molecule's total mass, may offer the molecule many pharmacological advantages. (Peptides C3-20, 21, 22); and

H. Various combinations of the changes described above. (Peptides C3-23, 24, 25, 27, 29, 31, 32).

In order to assay the factors in sections E and F, using the Arginine building unit, the alkyl arm was shortened. Using the Proline and Tic (Tetrahydroisoquinoline-3-carboxylic acid) building units, constraining conformational steric hindrance (using one or two rings) on the alkyl arm was introduced.

When assaying the deletion of some residues, a simple molecule was needed as a connecting arm forming the cyclization, since cyclization is crucial for conformational constraining. Several dicarboxylic acids long enough not to form a too small ring were chosen, thus totally altering the lead molecule's conformation. The dicarboxylic acids chosen were: pimelic acid—7 carbons long, 1,3-phenylenediacetic acid—7 carbons long and 1,4-phenylenedipropionic acid—10 carbons long. The last two have an aromatic ring aimed at reducing the conformational freedom of this connective arm.

### Synthesis of the C3 Peptide Analogs

The peptides were synthesized on an MBHA solid support using the “Tea bag” method. In some peptides (C3-1-15, C3-23-35, C3-29, C3-31) the building units were constructed directly on solid support. All peptides were cyclized by an amide bond according to a standard coupling procedure. At the end of the synthesis the peptides were cleaved off the solid support using TMSOTf:TFA. The peptides were purified on preparative HPLC using an R^{P}—C18 column in a water:acetonitrile gradient (programs 1,8) and characterized by MS. Their purity level was determined by analytical HPLC using an analytical R^{P}—C18 column in similar gradients. The structure of these peptide analogs is shown in Scheme 1:

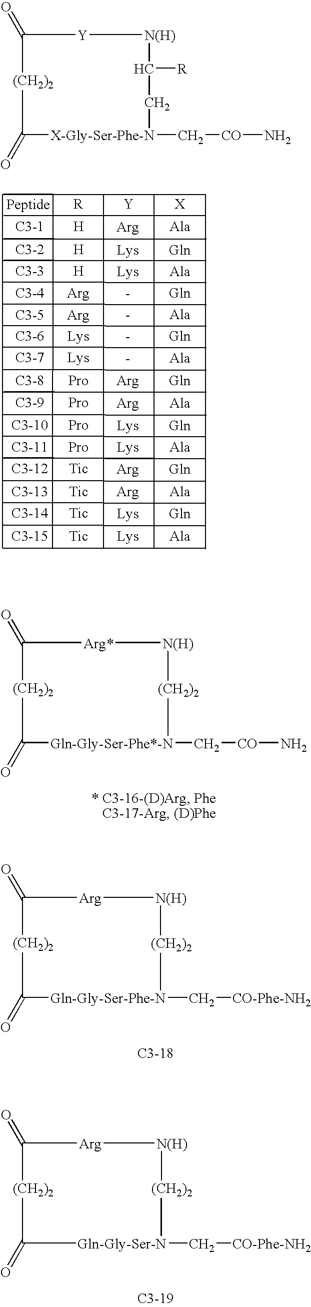

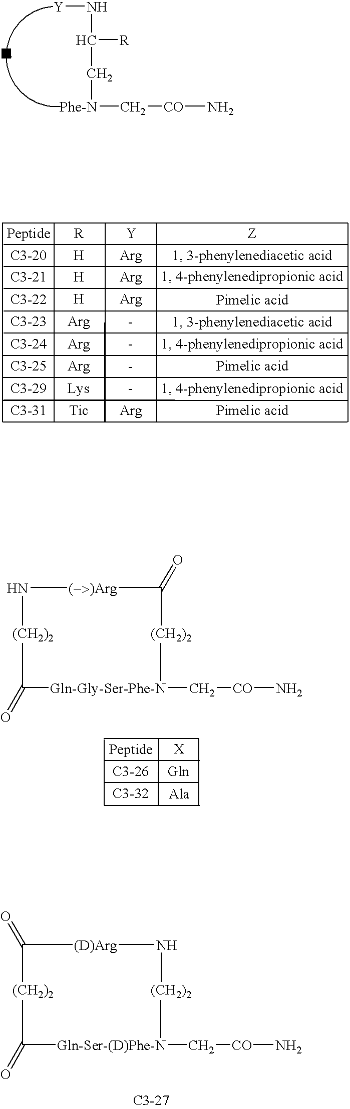

Specifically, the formula in the top left covering peptides C3-1 to C3-15 (SEQ ID NO:2), the formula in the bottom left covering peptides C3-20 to C3-25, C3-29 and C3-31 (SEQ ID NO:3), the formula covering peptides C3-16 and C3-17 (SEQ ID NO:4 and 5), the formula covering peptide C3-18 (SEQ ID NO:6), the formula covering peptide C3-19 (SEQ ID NO:7), the formula covering peptides C3-26 and C3-32 (SEQ ID NO:8) and the formula covering peptide C3-27 (SEQ ID NO:9). The present invention is directed to the compositions and methods described above using each of these formulas. The results of this synthesis are presented in Table 2:

TABLE 2
Synthesis results, purity and characterization of the C3 peptides.

| | Purity^{a} | Calc. | Found | Net Wt. | |
|---|---|---|---|---|---|
| Peptide | (%) | MW^{b} | MW^{b} | (mg) | Yield (%) |
| C3-1 | 89 | 717.8 | 718.8 | 2.8 | 2.7 |
| C3-2 | 96 | 746.8 | 747.5 | 4.4 | 4.2 |
| C3-3 | 60 | 689.8 | 690.6 | 3.4 | 3.5 |
| C3-4 | 39 | 717.8 | 718.5 | 7.8 | 7.6 |
| C3-5 | 100 | 660.7 | 661.5 | 1.5 | 1.6 |
| C3-6 | 25 | 689.8 | 690.4 | 12.8 | 13 |
| C3-7 | 31 | 632.7 | 633.4 | 9.8 | 10.9 |
| C3-8 | 53 | 814.9 | 815.5 | 5.6 | 4.9 |
| C3-9 | 87 | 757.9 | 758.5 | 4.5 | 4.1 |
| C3-10 | 52 | 786.9 | 787.4 | 8.2 | 7.3 |
| C3-11 | 37 | 729.8 | 730.4 | 6.4 | 6.1 |
| C3-12 | 92 | 878 | 877.5 | 1.9 | 1.5 |
| C3-13 | 61 | 820.9 | 820.5 | 2.5 | 2.2 |
| C3-14 | 69 | 845 | 849.5 | 2.7 | 2.2 |
| C3-15 | 94 | 792.9 | 792.4 | 3.3 | 2.9 |
| C3-16 | 62 | 774.8 | 775.1 | 1.4 | 1.2 |
| C3-17 | 74 | 774.8 | 775.6 | 2 | 1.8 |
| C3-18 | 100 | 922 | 922.3 | 0.8 | 0.6 |
| C3-19 | 80 | 774.8 | 775.8 | 2.3 | 2 |
| C3-20 | 70 | 578.7 | 579.5 | 1.7 | 2.1 |
| C3-21 | 55 | 606.7 | 607.8 | 1.6 | 1.9 |
| C3-22 | 31 | 544.7 | 546 | 1.6 | 2.1 |
| C3-23 | 54 | 521.6 | 522.5 | 0.5 | 0.6 |
| C3-24 | 100 | 549.7 | 550.4 | 0.1 | 0.1 |
| C3-25 | 100 | 487.6 | 488.4 | 0.2 | 0.3 |
| C3-26 | 36 | 774.8 | 776 | 3.8 | 3.5 |
| C3-27 | 97 | 706.8 | 707.5 | 1.3 | 1.3 |
| C3-29 | 50 | 521.7 | 522.4 | 1.8 | 2.4 |
| C3-31 | 100 | 647.8 | 647.5 | 1.8 | 1.9 |
| C3-32 | 56 | 717.8 | 718.5 | 0.6 | 0.6 |

^{a}Determined by analytical HPLC
^{b}g/mole.

### Biological Activity Results of C3 Peptides

### Cell Infection Inhibition Assay

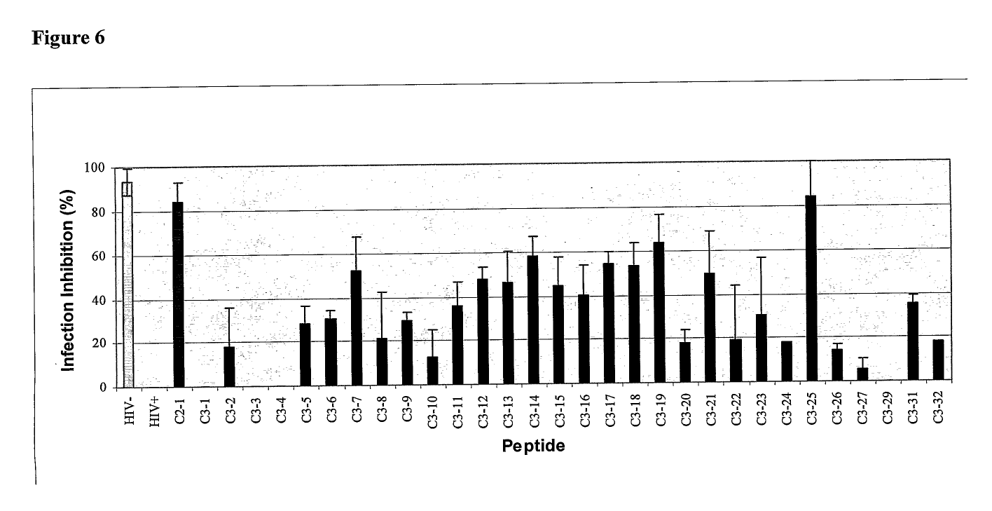

The assay was conducted as discussed above and the results are presented in FIG. 6. This figure includes, for comparison, the inhibition of the lead peptide C2-1, as determined in the cell infection inhibition assay.

Most peptides showed inhibitory activity. The most active peptide was C3-25, reaching 84±16% inhibition. This degree of inhibition is a little higher than that of the lead peptide C2-1, since its activity was measured at 129 μM while C3-25 reached its activity at 100 μM only. Another quite active peptide was C3-19 (64±12% inhibition).

### Example 3

### Design, Synthesis and Activity of the C4 Peptides

Another set of peptide analogs C4 was designed based on the most active compound C3-25. This set of compounds contains 20 backbone cyclic analogs (C4-1 to C4-20) comprising one aromatic side chain (R1) and one positively charged side chain (R4) as indicated in Scheme 2 and table 3.

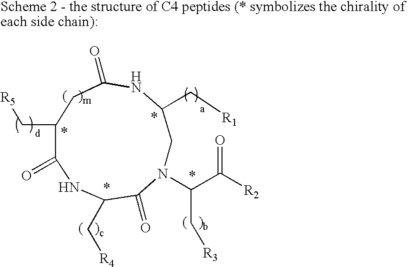

TABLE 3
members of the C4 set

| Peptide | R1 | a | * | R2 | R3 | b | * | R4 | c | * | R5 | d | * | m |
|---|---|---|---|---|---|---|---|---|---|---|---|---|---|---|
| C3-25 | guanidino | 2 | L | NH₂ | H | 0 | — | Ph | 1 | L | H | 0 | — | 6 |
| C4-1 | Ph | 1 | L | NH₂ | H | 0 | — | guanidino | 2 | L | H | 0 | — | 2 |
| C4-2 | Ph | 1 | L | NH₂ | H | 0 | — | guanidino | 2 | L | H | 0 | — | 3 |
| C4-3 | Ph | 1 | L | NH₂ | H | 0 | — | guanidino | 2 | L | H | 0 | — | 4 |
| C4-4 | Ph | 1 | L | NH₂ | H | 0 | — | guanidino | 2 | L | H | 0 | — | 5 |
| C4-5 | Ph | 1 | L | NH₂ | H | 0 | — | guanidino | 2 | L | H | 0 | — | 6 |
| C4-6 | Ph | 1 | L | NH₂ | H | 0 | — | guanidino | 2 | D | H | 0 | — | 2 |
| C4-7 | Ph | 1 | L | NH₂ | H | 0 | — | guanidino | 2 | D | H | 0 | — | 3 |
| C4-8 | Ph | 1 | L | NH₂ | H | 0 | — | guanidino | 2 | D | H | 0 | — | 4 |
| C4-9 | Ph | 1 | L | NH₂ | H | 0 | — | guanidino | 2 | D | H | 0 | — | 5 |
| C4-10 | Ph | 1 | L | NH₂ | H | 0 | — | guanidino | 2 | D | H | 0 | — | 6 |
| C4-11 | Ph | 1 | D | NH₂ | H | 0 | — | guanidino | 2 | L | H | 0 | — | 2 |
| C4-12 | Ph | 1 | D | NH₂ | H | 0 | — | guanidino | 2 | L | H | 0 | — | 3 |
| C4-13 | Ph | 1 | D | NH₂ | H | 0 | — | guanidino | 2 | L | H | 0 | — | 4 |
| C4-14 | Ph | 1 | D | NH₂ | H | 0 | — | guanidino | 2 | L | H | 0 | — | 5 |
| C4-15 | Ph | 1 | D | NH₂ | H | 0 | — | guanidino | 2 | L | H | 0 | — | 6 |
| C4-16 | Ph | 1 | D | NH₂ | H | 0 | — | guanidino | 2 | D | H | 0 | — | 2 |
| C4-17 | Ph | 1 | D | NH₂ | H | 0 | — | guanidino | 2 | D | H | 0 | — | 3 |
| C4-18 | Ph | 1 | D | NH₂ | H | 0 | — | guanidino | 2 | D | H | 0 | — | 4 |
| C4-19 | Ph | 1 | D | NH₂ | H | 0 | — | guanidino | 2 | D | H | 0 | — | 5 |
| C4-20 | Ph | 1 | D | NH₂ | H | 0 | — | guanidino | 2 | D | H | 0 | — | 6 |

1.-19. (canceled)

20. A backbone cyclized CD4 mimetic comprising a peptide sequence of three to twelve amino acids that incorporates at least one building unit, said building unit, containing one nitrogen atom of the peptide backbone connected to a bridging group comprising a disulfide, amide, thioether, thioester, imine, ether, or alkene bridge, wherein at least one building unit is connected via the bridging group to a moiety selected from the group consisting of a second building unit, a side chain of an amino acid residue of the peptide sequence, and a N-terminal amino acid residue, to form a cyclic structure.

21. The CD4 mimetic of claim 20 wherein the bridging group is a chemical linker having the General Formula I:
—(CH)ₙ—(CH)Y-M-A-B-
wherein n is an integer for 1 to 8; M is selected from the group consisting of a disulfide, amide, thioether, thioester, imine, ether, or alkene bridge; Y is hydrogen or an amino acid side chain; A is (CH₂)ₘ wherein m is an integer for 1 to 8, or C(R)—NH wherein R is an amino acid side chain; and B is absent or is the residue of a molecule comprising two carboxylic groups.

22. The CD4 mimetic of claim 20 having the general Formula II: 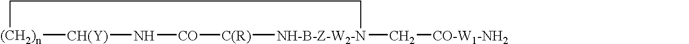
wherein:
Y is hydrogen or is the side chain of Arg, DArg, Lys, DLys, Phe, DPhe, Tic, DTic, Pro, or DPro;
n is 1 to 5;
R is the side chain of Arg, Lys, Phe, DArg, DLys or DPhe;
W₁ is absent or is Phe;
W₂ is absent or is Phe, DPhe, Arg or DArg
Z is 0 to 3 amino acid residues; and
B is the residue of a molecule comprising two carboxylic groups or is absent.

23. The CD4 mimetic of claim 30 having a Formula selected from the group consisting of Formulae III-XII:

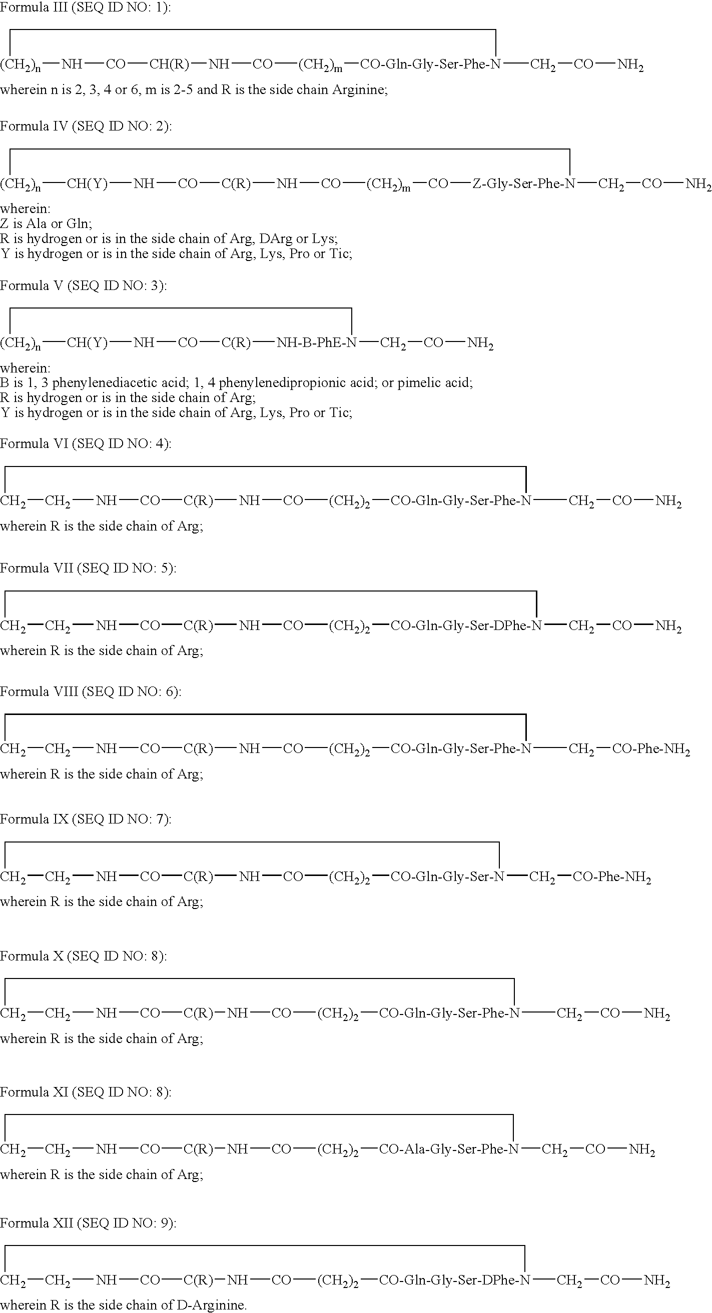

24. The CD4 mimetic of claim 20 having the general Formula XIII: 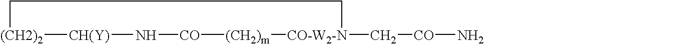
wherein:
Y is the side chain of Arg, Phe or DPhe;
W₂ is Phe, Arg or DArg; and
m is 2-6.

25. The CD4 mimetic of claim 20 selected from the group consisting of:

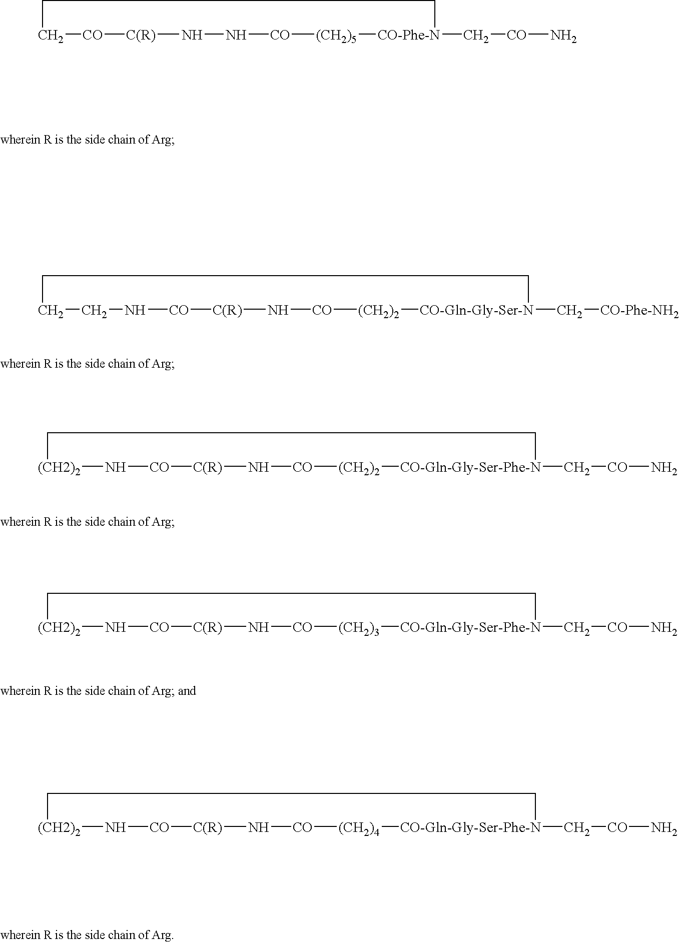

26. A pharmaceutical composition comprising a pharmaceutically acceptable carrier and as an active ingredient a backbone cyclized CD4 mimetic comprising a peptide sequence of three to twelve amino acids that incorporates at least one building unit, said building unit, containing one nitrogen atom of the peptide backbone connected to a bridging group comprising a disulfide, amide, thioether, thioester, imine, ether, or alkene bridge, wherein at least one building unit is connected via the bridging group to a moiety selected from the group consisting of a second building unit, a side chain of an amino acid residue of the peptide sequence, and a N-terminal amino acid residue, to form a cyclic structure.

27. The pharmaceutical composition of claim 26 wherein the bridging group is a chemical linker having the General Formula I:
—(CH)ₙ—(CH)Y-M-A-B-
wherein n is an integer for 1 to 8; M is selected from the group consisting of a disulfide, amide, thioether, thioester, imine, ether, or alkene bridge; Y is hydrogen or an amino acid side chain; A is (CH₂)ₘ wherein m is an integer for 1 to 8, or C(R)—NH wherein R is an amino acid side chain;
and B is absent or is the residue of a molecule comprising two carboxylic groups.

28. The pharmaceutical composition of claim 26 wherein the backbone cyclized CD4 mimetic is of general Formula II (SEQ ID NO: 1): 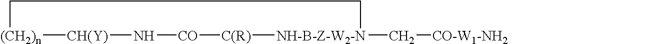
wherein:
Y is hydrogen or is the side chain of Arg, DArg, Lys, DLys, Phe, DPhe, Tic, DTic, Pro, or DPro;
n is 1 to 5;
R is the side chain of Arg, Lys, Phe, DArg, DLys or DPhe;
W₁ is absent or is Phe;
W₂ is absent or is Phe, DPhe, Arg or DArg
Z is 0 to 3 amino acid residues; and
B is the residue of a molecule comprising two carboxylic groups or is absent.

29. The pharmaceutical composition of claim 26 wherein the backbone cyclized CD4 mimetic is of a Formula selected from the group consisting of Formulae III-XII:

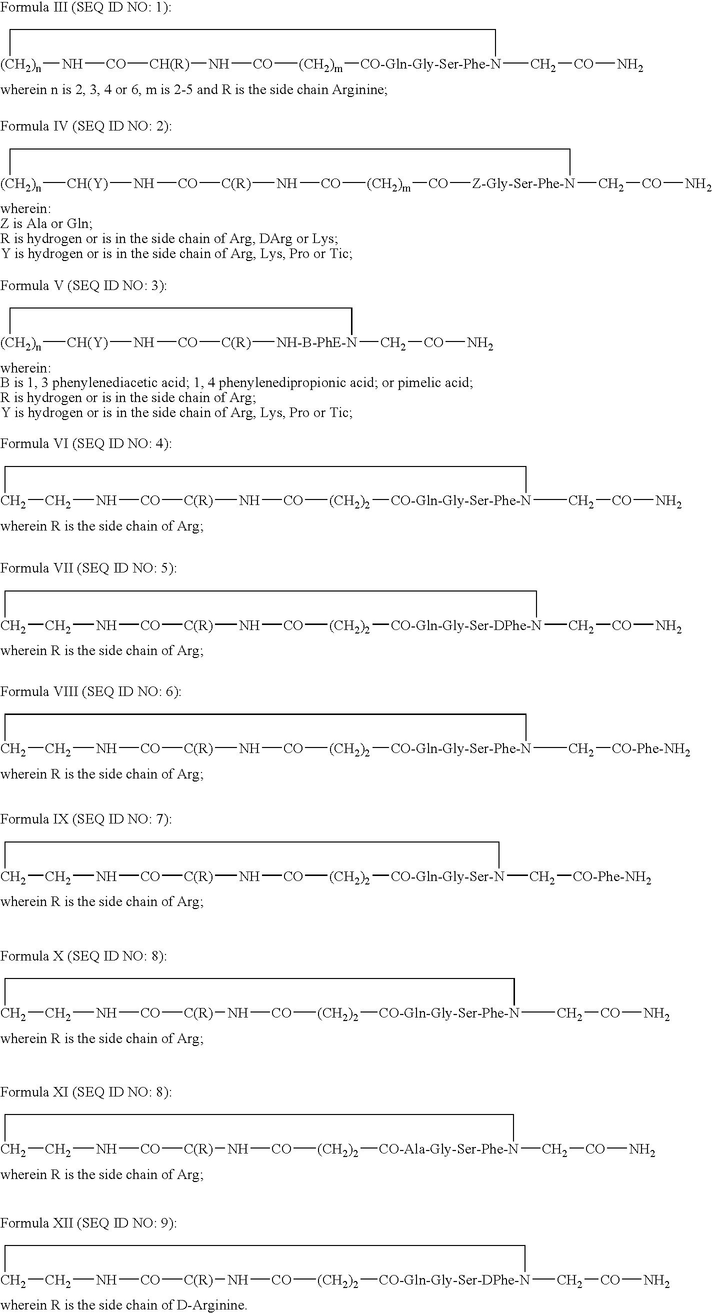

30. The pharmaceutical composition of claim 26 wherein the backbone cyclized CD4 mimetic is of general Formula XIII: 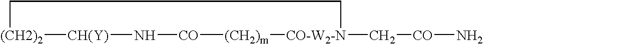
wherein:
Y is the side chain of Arg, Phe or DPhe;
W₂ is Phe, Arg or DArg; and
m is 2-6.

31. The pharmaceutical composition of claim 26 wherein the backbone cyclized CD4 mimetic is selected from the group consisting of:

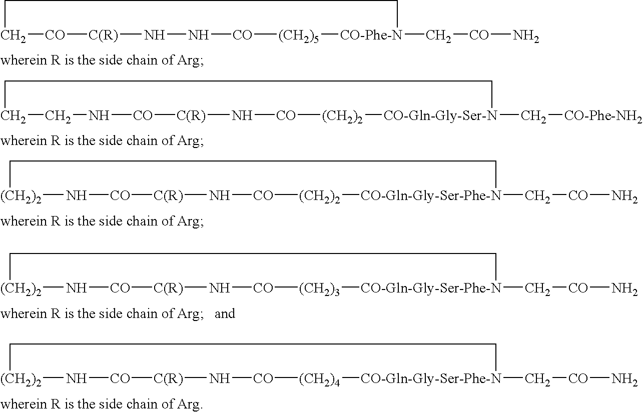

32. A method of treating a subject with HIV, comprising administering to the subject a therapeutically effective amount of backbone cyclized CD4 mimetic comprising a peptide sequence of three to twelve amino acids that incorporates at least one building unit, said building unit, containing one nitrogen atom of the peptide backbone connected to a bridging group comprising a disulfide, amide, thioether, thioester, imine, ether, or alkene bridge, wherein at least one building unit is connected via the bridging group to a moiety selected from the group consisting of a second building unit, a side chain of an amino acid residue of the peptide sequence, and a N-terminal amino acid residue, to form a cyclic structure.

33. The method of claim 32 wherein the bridging group is a chemical linker having the General Formula I:
—(CH)ₙ—(CH)Y-M-A-B-
wherein n is an integer for 1 to 8; M is selected from the group consisting of a disulfide, amide, thioether, thioester, imine, ether, or alkene bridge; Y is hydrogen or an amino acid side chain; A is (CH₂)ₘ wherein m is an integer for 1 to 8, or C(R)—NH wherein R is an amino acid side chain;
and B is absent or is the residue of a molecule comprising two carboxylic groups.

34. The method of claim 32 wherein the backbone cyclized CD4 mimetic is according to general Formula II (SEQ ID NO: 1): 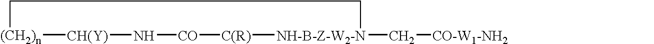
wherein:
Y is hydrogen or is the side chain of Arg, DArg, Lys, DLys, Phe, DPhe, Tic, DTic, Pro, or DPro;
n is 1 to 5;
R is the side chain of Arg, Lys, Phe, DArg, DLys or DPhe;
W₁ is absent or is Phe;
W₂ is absent or is Phe, DPhe, Arg or DArg
Z is 0 to 3 amino acid residues; and
B is the residue of a molecule comprising two carboxylic groups or is absent.

35. The method of claim 32 wherein the backbone cyclized CD4 mimetic is according to a Formula selected from the group consisting of Formulae III-XII:

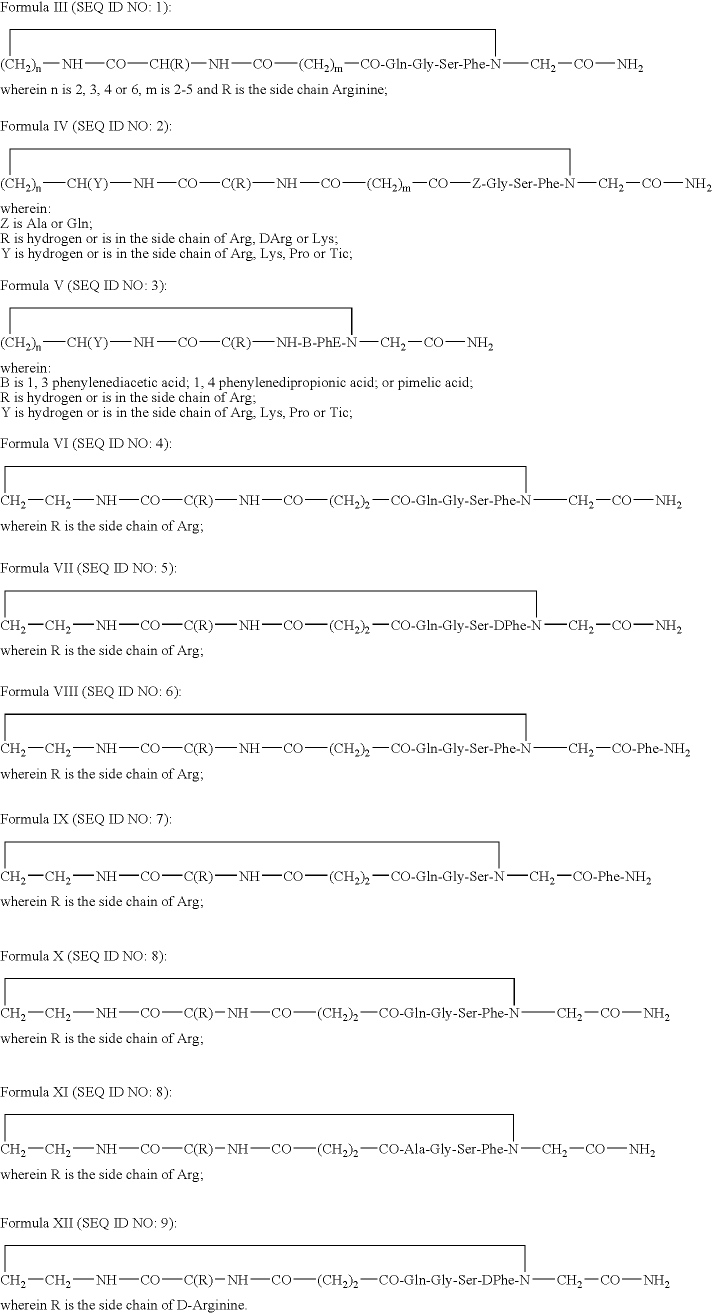

36. The method of claim 32 wherein the backbone cyclized CD4 mimetic is according to general Formula XIII: 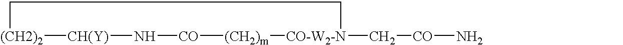
wherein:
Y is the side chain of Arg, Phe or DPhe;
W₂ is Phe, Arg or DArg; and
m is 2-6.

37. The method of claim 32 wherein the backbone cyclized CD4 mimetic is selected from the group consisting of: [chemical structure]


SEQUENCE LISTING (9 sequences)

SEQ ID NO: 1 - protein, 6 aa, Artificial
  Synthetic peptide analog
RQGSFG

SEQ ID NO: 2 - protein, 7 aa, Artificial
  Synthetic peptide analog
XXXGSFG

SEQ ID NO: 3 - protein, 4 aa, Artificial
  Synthetic peptide analog
XRFG

SEQ ID NO: 4 - protein, 6 aa, Artificial
  Synthetic peptide analog
XQGSFG

SEQ ID NO: 5 - protein, 6 aa, Artificial
  Synthetic peptide analog
RQGSXG

SEQ ID NO: 6 - protein, 7 aa, Artificial
  Synthetic peptide analog
RQGSFGF

SEQ ID NO: 7 - protein, 6 aa, Artificial
  Synthetic peptide analog
RQGSGF

SEQ ID NO: 8 - protein, 6 aa, Artificial
  Synthetic peptide analog
RXGSFG

SEQ ID NO: 9 - protein, 5 aa, Artificial
  Synthetic peptide analog
XGSXG